SEQUENCE LISTING (9 sequences)

SEQ ID NO: 1 - DNA, 17 bp, Homo sapiens
tctgcggcgttttatca

SEQ ID NO: 2 - DNA, 18 bp, Homo sapiens
gtgtctgcggcgttttat

SEQ ID NO: 3 - DNA, 23 bp, Homo sapiens
agactcgtggtggacttctctca

SEQ ID NO: 4 - DNA, 17 bp, Homo sapiens
acgggcaacataccttg

SEQ ID NO: 5 - DNA, 21 bp, Homo sapiens
ggcatagcagcaggatgmaga

SEQ ID NO: 6 - DNA, 27 bp, Homo sapiens
catcctgctgctatgcctcatcttctt

SEQ ID NO: 7 - DNA, 25 bp, Homo sapiens
tcctgctgctatgcctcatcttctt

SEQ ID NO: 8 - DNA, 26 bp, Homo sapiens
atcctgctgctatgcctcatcttctt

SEQ ID NO: 9 - DNA, 26 bp, Homo sapiens
tggatgtgtctgcggcgttttatcat

(19) United States
(12) Patent Application Publication
(10) Pub. No.: US 2010/0255482 A1
(43) Pub. Date: Oct. 7, 2010

(54) Hepatitis B Virus (HBV) Specific Oligonucleotide Sequences

(75) Inventors: Lu-ping Shen, San Leandro, CA (US); Carrie Li Wong, San Francisco, CA (US); Lailing Ku, Pleasanton, CA (US); Andy Ying, Fremont, CA (US); Charlene Bush-Donovan, Livermore, CA (US)

Correspondence Address: SIEMENS CORPORATION;INTELLECTUAL PROPERTY DEPARTMENT, 170 WOOD AVENUE SOUTH, ISELIN, NJ, 08830, US

(73) Assignee: SIEMENS HEALTHCARE DIAGNOSTICS INC., Tarrytown, NY (US)

(21) Appl. No.: 12/741,062

(22) PCT Filed: Oct. 29, 2008
(86) PCT No.: PCT/US2008/081491

§ 371 Date: May 3, 2010

Related U.S. Application Data
(60) Provisional application No. 60/985,824, filed on Nov. 6, 2007.

(51) Int. Cl. C12Q 1/68; C07H 21/04

(52) U.S. Cl. 435/6; 536/23.1; 536/24.3

(57) ABSTRACT

The present invention relates to oligonucleotide sequences for amplification primers and detection probes and to their use in nucleic acid amplification methods for the detection of HBV in biological samples. In particular, oligonucleotide sequences are provided for the sensitive qualitative or quantitative detection of all eight HBV genotypes. The invention also provides oligonucleotide primer sets and primer/probe sets in the form of kits for the diagnosis of HBV infection.

### BACKGROUND OF THE INVENTION

Hepatitis B virus (HBV), an important human pathogen, is a member of the orthohepadnavirus genus in the Hepadnaviridae family. All members of this family are small, hepatotropic DNA viruses which display a similar virion morphology and genome organization, and replicate via reverse transcription of an RNA intermediate. Four major serological subtypes have been found that have distinct geographical distributions, with some overlap. DNA sequencing has allowed replacement of the initial serotypic classification of HBV strains by a more systematic genotype system that currently consists of 8 members (genotypes A-H).

Infection with HBV is a global public health problem. HBV is endemic in the human population and hyperendemic in many regions of the world. Despite the availability of hepatitis B vaccine, the overall prevalence of HBV infection has only slightly declined in recent years. The World Health Organization (WHO) has estimated that more than one third of the world's population has been infected with HBV. In many cases, HBV infection is self-limited and asymptomatic, and the virus is believed to be eliminated by the immune system. However, about 5% of the human population are chronic carriers of HBV. Chronic HBV infection develops in approximately 90% of children infected at birth and 30%-60% of children infected between 1 to 5 years of age compared with 2%-6% of older children and adults. Chronic HBV infection may be asymptomatic for many years and/or result in only slight liver damages (C. Niederau et al., New Engl. J. Med., 1996, 334: 403-415); however in a certain number of cases (up to 30%), chronic HBV infection leads to liver diseases including cirrhosis, liver failure, and hepatocellular carcinoma, making HBV a major cause of morbidity and mortality (A. S. Lok et al., Hepatology, 2001, 34: 1225-1241; W. M. Lee et al., New Engl. J. Med., 1997, 337: 1735-1745; Loeb et al., Hepatology, 2000, 32: 626-629). In the United States, chronic HBV infection affects 1.25 million people (A. S. Lok et al., Hepatology, 2001, 34: 1225-1241) at a cost of more than $700 million annually.

HBV viral load has been shown to be indicative of the likelihood of liver injury, its intensity, and progression to cirrhosis and hepatocellular carcinoma (HCC). Thus, the course of HBV infection should preferably be defined individually for each patient being evaluated for clinical trials or treatment. Although progress has been made in the management of chronic HBV infection, none of the currently available treatments has so far resulted in a complete eradication of the virus in chronically infected patients. Today, the primary goal of therapy for HBV infection is to achieve sustained suppression of viral replication to levels that are associated with disappearance of intra-hepatic necrosis and inflammation in order to limit liver damage and reduce the probability of long-term complications of hepatic decompensation and HCC.

Thus, the diagnostic strategy in HBV infection is to identify patients who are currently infected with HBV, determine if they have active viral replication and ongoing liver damage, and assess if they are appropriate candidates for treatment. Several diagnostic tools can be used to detect HBV and/or quantify and/or monitor the status of HBV infection in patients. These include serological, virological, biochemical, and histological tests and assays.

Immunological tests are performed by demonstration of viral antigens or their respective antibodies in serum. At least three distinct antigen-antibody systems are intimately related to HBV: hepatitis B surface antigen (HBsAg), which appears in the sera of most patients during the late stage of the incubation period, persists during the acute illness, and sharply decreases when antibodies to the surface antigen become detectable; hepatitis B core antigen (HBcAg), which is associated with the viral core; and hepatitis B “e” antigen (HBeAg), which is secreted by infected hepatocytes and serves as a marker for active viral replication. However, in addition to being laborious and time-consuming, these immunological methods lack sensitivity as screening assays and may generate false negatives. In particular, when an immunological method is carried out within the sero-conversion phase of a patient, HBV infection may remain undetected. Additionally or alternatively, genetic mutations in the gene(s) encoding the HBV antigen(s) recognized by such immunological tests may result in loss of immunoreactivity, leading to false negatives.

Nucleic acid amplification tests (NATs) for the detection of HBV have been developed based on target amplification (e.g., PCR and PCR-derived methods such as real-time PCR) and signal amplification methods (see, for example, Urdea et al., Gene, 1987, 61: 253-264; Kaneko et al., Proc. Natl. Sci. U.S.A., 1989, 86: 312-316; Sumazaki et al., J. Med. Virol., 1989, 27: 304-308; Theilman et al., Liver, 1989, 9: 322-328; Liang et al., Hepatology, 1990, 12: 204-212; Lo et al., J. Clin. Microbiol., 1990, 28: 1411-1416; Fiordalisi et al., J. Med. Virol., 1990, 31: 297-300; Pasquinelli et al., J. Med. Virol., 1990, 31: 135-140; Brunetto et al., Proc. Natl. Acad. Sci., USA, 1991, 88: 4186-4190; Brunetto et al., Prog. Clin. Biol. Res., 1991, 364: 211-216; Saito et al., J. Med. Virol., 1999, 58: 325-331; Mercier et al., J. Virol Methods, 1999, 77: 1-9; Loeb et al., Hepatology, 2000, 32: 626-629; Pas et al., J. Clin Microbiol., 2000, 38: 2897-2901; Drosten et al., Transfusion, 2000, 40: 718-724; Weinberger et al., J. Virol Methods, 2000, 85: 75-82; Chen et al., J. Med. Virol., 2001, 65: 250-256; Meng et al., J. Clin Microbiol., 2001, 39: 2937-2945; U.S. Pat. Nos. 5,614,362; 5,736,316; 5,736,334; 5,780,219; 5,858,652; 5,955,598; 6,583,279; 6,635,428; and 7,015,317; U.S. Appln. Nos. 2003-0143527; 2003-0215790; 2004-0029111; 2004-0191776; 2005-0037414; and 2005-0175990; International Appln. Nos. WO 9013667 and WO 05061737; and European Appln. No. EP 0 860 505 A).

Several diagnostic kits have been marketed in this field including, for example, Versant® HBV DNA (bDNA, Bayer Diagnostics), Amplicor HBV Monitor® and CobasAmplicor HBV Monitor® (Roche Molecular Systems), and Digene Hybrid-Capture™ 2 HBV DNA test (Digene Corporation).

Although existing nucleic acid amplification assays for the detection of HBV provide high specificity and sensitivity and require only short processing time, they exhibit certain disadvantages and limitations. Some of the main concerns include the inability to detect all genotypes of HBV with equal efficiency, and inability to accurately detect and quantify HBV from very low to very high concentrations without combining at least two tests or performing additional assays. Additionally or alternatively, such existing amplification assays may fail to detect certain HBV genetic variants, e.g., HBSAg and/or core mutants. Clearly, the development of improved nucleic acid amplification assays for the detection of HBV infection remains highly desirable.

### SUMMARY OF THE INVENTION

The present invention is directed to systems for the rapid, selective, and specific detection and quantification of hepatitis B virus (HBV) in biological samples. In particular, the invention encompasses reagents that can be used for developing nucleic acid amplification tests for the detection of HBV nucleic acids and the diagnosis of HBV infection. More specifically, the invention provides oligonucleotide sequences for amplification primers and detection probes that are useful for the detection of target nucleic acid sequences within the HBV surface antigen gene. In certain embodiments, the inventive oligonucleotide sequences have the advantage of recognizing all eight genotypes (A-H) of HBV, and allowing for a very wide range of quantification.

In one aspect, the present invention provides isolated oligonucleotides having a sequence selected from the group consisting of SEQ ID NOs. 1-9 (as listed in the table presented in FIG. 1), complementary sequences thereof, active fragments thereof, and combinations thereof. In particular, in some embodiments, isolated oligonucleotides having a sequence selected from the group consisting of SEQ ID NOs. 1-5, complementary sequences thereof, active fragments thereof, and combinations thereof, are used as amplification primers. In some embodiments, isolated oligonucleotides having a sequence selected from the group consisting of SEQ ID NOs. 6-9, complementary sequences thereof, active fragments thereof, and combinations thereof, are used as detection probes.

In certain embodiments, isolated oligonucleotides used as detection probes comprise a detectable label, e.g., a fluorescent moiety attached at the 5′ end of the oligonucleotide. Such oligonucleotides may further comprise a quencher moiety attached to its 3′ end. For example, an isolated oligonucleotide detection probe can comprise 6-carboxyfluorescein attached at its 5′ end and a Black Hole Quencher at its 3′ end.

In another aspect, the present invention provides a collection of oligonucleotides for detecting HBV in a test sample. The collection comprises at least one primer set selected from the group consisting of Primer Set 1, Primer Set 2, and Primer Set 3, wherein:
Primer Set 1 comprises a forward primer having a sequence as set forth in SEQ ID NO. 1 or any active fragment thereof, and a reverse primer having a sequence as set forth in SEQ ID NO. 4 or any active fragment thereof;
Primer Set 2 comprises a forward primer having a sequence as set forth in SEQ ID NO. 2 or any active fragment thereof, and a reverse primer having a sequence as set forth in SEQ ID NO. 4 or any active fragment thereof; and
Primer Set 3 comprises a forward primer having a sequence as set forth in SEQ ID NO. 3 or any active fragment thereof, and a reverse primer having a sequence as set forth in SEQ ID NO. 5 or any active fragment thereof.

In another aspect, the present invention provides a collection of oligonucleotides for detecting HBV in a test sample, that comprises at least one primer/probe set selected from the group consisting of: Primer/Probe Set 1(a), Primer/Probe Set 1(b), Primer/Probe Set 1(c), Primer/Probe Set 2(a), Primer/Probe Set 2(b), Primer/Probe Set 2(c), and Primer/Probe Set 3, wherein:
Primer/Probe Set 1(a) comprises a forward primer having a sequence as set forth in SEQ ID NO. 1 or any active fragment thereof, a reverse primer having a sequence as set forth in SEQ ID NO. 4 or any active fragment thereof, and a detection probe having a sequence as set forth in SEQ ID NO. 6 or any active fragment thereof;
Primer/Probe Set 1(b) comprises a forward primer having a sequence as set forth in SEQ ID NO. 1 or any active fragment thereof, a reverse primer having a sequence as set forth in SEQ ID NO. 4 or any active fragment thereof, and a detection probe having a sequence as set forth in SEQ ID NO. 7 or any active fragment thereof;
Primer/Probe Set 1(c) comprises a forward primer having a sequence as set forth in SEQ ID NO. 1 or any active fragment thereof, a reverse primer having a sequence as set forth in SEQ ID NO. 4 or any active fragment thereof, and a detection probe having a sequence as set forth in SEQ ID NO. 8 or any active fragment thereof;
Primer/Probe Set 2(a), comprises a forward primer having a sequence as set forth in SEQ ID NO. 2 or any active fragment thereof, a reverse primer having a sequence as set forth in SEQ ID NO. 4 or any active fragment thereof, and a detection probe having a sequence as set forth in SEQ ID NO. 6 or any active fragment thereof;
Primer/Probe Set 2(b) comprises a forward primer having a sequence as set forth in SEQ ID NO. 2 or any active fragment thereof, a reverse primer having a sequence as set forth in SEQ ID NO. 4 or any active fragment thereof, and a detection probe having a sequence as set forth in SEQ ID NO. 7 or any active fragment thereof;
Primer/Probe Set 2(c) comprises a forward primer having a sequence as set forth in SEQ ID NO. 2 or any active fragment thereof, a reverse primer having a sequence as set forth in SEQ ID NO. 4 or any active fragment thereof, and a detection probe having a sequence as set forth in SEQ ID NO. 8 or any active fragment thereof; and
Primer/Probe Set 3 comprises a forward primer having a sequence as set forth in SEQ ID NO. 3 or any active fragment thereof, a reverse primer having a sequence as set forth in SEQ ID NO. 5 or any active fragment thereof, and a detection probe having a sequence as set forth in SEQ ID NO. 9 or any active fragment thereof.

In certain embodiments, the detection probe in the primer/probe set comprises a detectable label, such as a fluorescent moiety attached at the 5′ end of the detection probe. In some embodiments, the detection probe further comprises a quencher moiety attached at its 3′ end. For example, a detection probe in the primer/probe set may comprise 6-carboxyfluorescein attached at its 5′ end and a Black Hole Quencher attached at its 3′ end.

In yet another aspect, the present invention provides a kits for detecting HBV in a test sample. In certain embodiments, the kit comprises amplification reagents, and at least one of Primer Set 1, Primer Set 2, and Primer Set 3 described herein. In other embodiments, the kits comprises amplification reagents, and at least one of Primer/Probe Set 1(a), Primer/Probe Set 1(b), Primer/Probe Set 1(c), Primer/Probe Set 2(a), Primer/Probe Set 2(b), Primer/Probe Set 2(c), and Primer/Probe Set 3 described herein. The detection probes in the kit may be labeled as described herein.

In still another aspect, the present invention provides methods for detecting HBV in a test sample.

In certain embodiments, the method comprises steps of: providing a test sample suspected of comprising a HBV nucleic acid; contacting the test sample with at least one isolated oligonucleotide such that the at least one oligonucleotide can hybridized to the HBV nucleic acid, if present in the test sample; and detecting any oligonucleotide hybridized to the HBV nucleic acid, wherein detection of any oligonucleotide hybridized to the HBV nucleic acid indicates the presence of HBV in the test sample. In this method, the isolated oligonucleotide has a sequence selected from the group consisting of SEQ ID NOs. 1-9, complementary sequences thereof, active fragments thereof, and combinations thereof.

In other embodiments, the method comprises steps of: providing a test sample suspected of comprising a HBV nucleic acid; contacting the test sample with at least one primer set under conditions such that all or part of the HBV nucleic acid is amplified, if present in the test sample, thereby generating HBV amplicons; and detecting any HBV amplicons, wherein detection of HBV amplicons indicates the presence of HBV in the test sample. In this method, the primer set is selected from the group consisting of Primer Set 1, Primer Set 2, Primer Set 3, and combinations thereof, as described herein.

In yet other embodiments, the method comprises steps of: providing a test sample suspected of comprising a HBV nucleic acid; contacting the test sample with at least one primer/probe set under conditions such that all or part of the HBV nucleic acid is amplified, if present in the test sample, thereby generating HBV amplicons; and detecting any HBV amplicons using the detection probe of the primer/probe set, wherein the detection of the HBV amplicons is indicative of the presence of HBV in the test sample. In this method, the primer/probe set is selected from the group consisting of Primer/Probe Set 1(a), Primer/Probe Set 1(b), Primer/Probe Set 1(c), Primer/Probe Set 2(a), Primer/Probe Set 2(b), Primer/Probe Set 2(c), Primer/Probe Set 3, and combinations thereof, as described herein.

In certain methods of HBV detection of the present invention, the step of amplifying may be performed using a polymerase chain reaction (PCR), a Reverse-Transcriptase PCR(RT-PCR), or a Taq-Man™ assay. In methods where at least one primer/probe set is used, the detection probe of the primer/probe set may comprise a detectable label, such as a fluorescent moiety attached at its 5′ end. The detection probe may further comprises a quencher moiety attached at its 3′ end. For example, the detection probe may comprise 6-carboxyfluorescein attached at its 5′ end and a Black Hole Quencher attached at its 3′ end.

In certain embodiments, the test sample used in methods of HBV detection of the present invention, comprises or is derived from a bodily fluid or tissue selected from the group consisting of blood, serum, plasma, urine, seminal fluid, saliva, ocular lens fluid, lymphatic fluid, endocervical, urethral, rectal, vaginal, vulva-vaginal, nasopharyngeal, and liver samples. For example, the bodily fluid may be obtained from an individual suspected of being infected with HBV.

In certain embodiments, the methods of the invention may further comprise steps of: quantifying any HBV amplicons to determine the test sample's viral load; and providing a HBV diagnosis for the individual tested based on the viral load determined. Providing a HBV diagnosis may comprise one or more of: determining if the individual is infected with HBV, determining a HBV infection stage for the individual, determining if the individual is afflicted with a HBV disease, determining the severity of a HBV disease afflicting the individual, determining the progression of a HBV disease afflicting the individual, determining the likelihood that the individual has to develop a HBV disease, and determining the efficacy of a HBV therapy undergone by the individual. The HBV disease may be acute hepatitis, chronic hepatitis, cirrhosis, liver failure, or hepatocellular carcinoma.

In certain embodiments, methods of the present invention comprise monitoring viral progression and/or response to therapy when a patient is co-infected with a second disease (e.g., HIV and/or HCV) in addition to HBV. For example, a patient co-infected with a second disease such as HIV and/or HCV may receive therapy (e.g., pharmaceuticals, vaccines, other biological agents, etc.) in order to treat or control the second disease. Such therapy may affect the progression and/or response to therapy of HBV, which may be advantageously monitored using one or more methods of the present invention.

In certain embodiments, methods of the present invention comprise monitoring recurrence of HBV after the patient's immune system has been compromised. For example, a patient may undergo a procedure such as organ transplantation wherein it is advantageous to either temorarily or permanently compromise the immune system. In such instances, such a patient will have a greater chance of being infected with an opportunistic pathogen. Additionally or alternatively, any pathogen that exists in such a patient (e.g., a pathogen that is present at low levels and is controlled by the patient's immune system) will have a greater chance of expanding, resulting in recurrence of any disease or condition caused by such a pathogen. Methods of the present invention are advantageous in monitoring such recurrence.

In some embodiments, the methods of the invention may further comprise steps of: quantifying any HBV amplicons to determine the test sample's viral load; and selecting a therapy for the individual based on the viral load determined.

These and other objects, advantages and features of the present invention will become apparent to those of ordinary skill in the art having read the following detailed description of the preferred embodiments.

### BRIEF DESCRIPTION OF THE DRAWING

FIG. 1 is a table showing examples of inventive HBV specific oligonucleotide sequences for amplification primers and detection probes, that were derived from the HBV surface antigen gene. The length and Tm of each oligonucleotide sequence is also indicated in the table. The degenerate nucleotide designation “M” refers to the fact that the nucleotide at this position may be either A or C.

FIG. 2 is a table showing the size of the amplicons formed using different combinations of forward primer, reverse primer and probe.

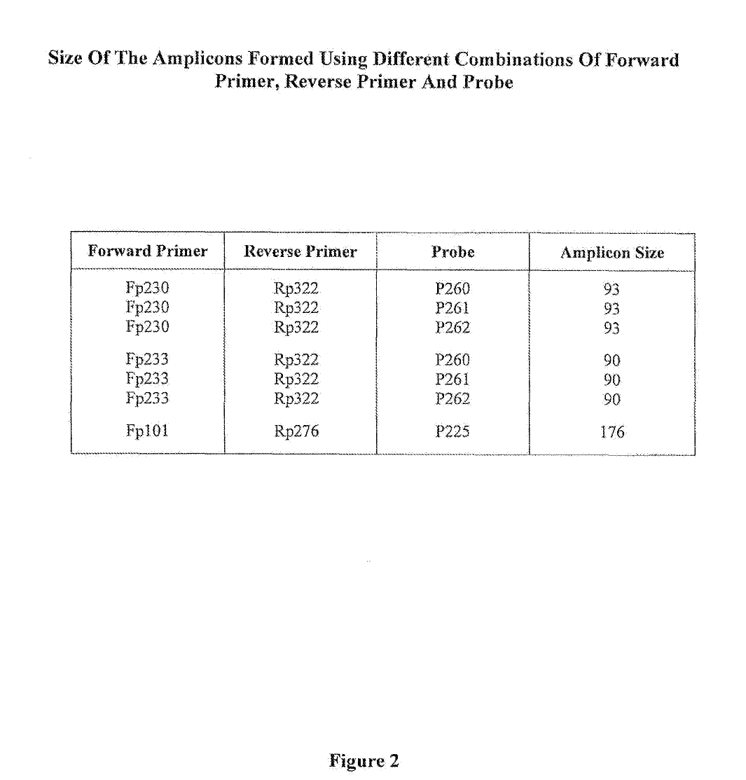

FIG. 3 is a graph which shows that the inventive oligonucleotide sequences recognize all eight HBV genotypes (A-H) with equal efficiency (see Example 1 for details).

FIG. 4 is a graph showing the linearity of quantitation detection of HBV recombinant DNA fragment (rDNA) by an HBV assay of the present invention (see Example 2 for details).

FIG. 5 is a table showing results of the DNA panel quantitation linearity study described in Example 2.

FIG. 6 is a table showing results of the quantitation detection limits (LoD) of an inventive HBV assay using HBV recombinant DNA fragments as target (see Example 2). Total N refers to the total number of detection experiments performed (replicates). # Detected refers to the number of times the HBV recombinant DNA was detected. Percent (%) is the percent of successful detections for the detection experiments (replicates) and is calculated by dividing # Detected by Total N.

FIG. 7 is a graph showing results of the quantitation detection limits (LoD) of an inventive HBV assay using HBV recombinant DNA fragments as target (see Example 2).

FIG. 8 is a graph showing the linearity of quantitation detection of HBV clinical specimens by an HBV assay of the present invention (see Example 4 for details).

FIG. 9 is a table showing results of the quantitation detection limits (LoD) of an inventive HBV assay for HBV clinical specimens (see Example 4). Total N refers to the total number of detection experiments performed (replicates). # Detected refers to the number of times the HBV virus was detected. Percent (%) is the percent of successful detections for the detection experiments (replicates) and is calculated by dividing # Detected by Total N.

FIG. 10 is a graph showing results of the quantitation detection limits (LoD) of an inventive HBV assay for HBV clinical specimens (see Example 4).

### DEFINITIONS

Throughout the specification, several terms are employed that are defined in the following paragraphs.

The terms “individual”, “subject”, and “patient” are used herein interchangeably. They refer to a human being that can be the host of hepatitis B virus (HBV), but may or may not be infected with the virus, and/or may or may not have a HBV disease. The terms do not denote a particular age, and thus encompass adults, children, newborns, as well as fetuses.

As used herein, the term “HBV disease” refers to any disease or disorder known or suspected to be associated with and/or caused, directly or indirectly, by HBV. HBV diseases include, but are not limited to, a wide variety of liver disease, such as subclinical carrier state to acute hepatitis, chronic hepatitis, cirrhosis, and hepatocellular carcinoma. HBV is also known to be associated with several primarily non-hepatic disorders including polyarteritis nodosa and other collagen vascular diseases, membranous glomerulonephritis, essential mixed cryoglobulinemia, and popular acrodermatitis of childhood.

The term “test sample”, as used herein, refers to any liquid or solid material suspected of containing HBV. A test sample may be, or may be derived from, any biological tissue or fluid that can contain HBV nucleic acids. Frequently, the sample will be a “clinical sample”, i.e., a sample obtained or isolated from a patient to be tested for HBV infection. Such samples include, but are not limited to, bodily fluids which contain cellular materials and may or may not contain cells, e.g., blood, blood product, plasma, serum, urine, seminal fluid, saliva, lymphatic fluid, amniotic fluid, synovial fluid, cerebrospinal fluid, peritoneal fluid, and the like; endocervical, urethral, rectal, vaginal, vulva-vaginal samples; and archival samples with known diagnosis. Test samples may include sections of tissue (e.g., liver biopsy samples), such as frozen sections. The term “test sample” also encompasses any material derived from processing a biological sample. Derived materials include, but are not limited to, cells (or their progeny) isolated from the sample, cell components, and nucleic acid molecules extracted from the sample. Processing of a biological sample to obtain a test sample may involve one of more of: filtration, distillation, centrifugation, extraction, concentration, dilution, purification, inactivation of interfering components, addition of reagents, and the like.

The terms “nucleic acid”, “nucleic acid molecule”, and “polynucleotide” are used herein interchangeably. They refer to a deoxyribonucleotide or ribonucleotide polymer in either single- or double-stranded form, and unless otherwise stated, encompass nucleic acid polymer comprising analogs of natural nucleotides that can function in a similar manner as naturally-occurring nucleotides.

The term “oligonucleotide”, as used herein, refers to a string of nucleotides or analogs thereof. Oligonucleotides may be obtained by a number of methods including, for example, chemical synthesis, restriction enzyme digestion or PCR. As will be appreciated by one skilled in the art, the length of an oligonucleotide (i.e., the number of nucleotides) can vary widely, often depending on the intended function or use of the oligonucleotide. Generally, oligonucleotides comprise between about 5 and about 300 nucleotides, for example, between about 15 and about 200 nucleotides, between about 15 and about 100 nucleotides, or between about 15 and about 50 nucleotides. Throughout the specification, whenever an oligonucleotide is represented by a sequence of letters (chosen from the four base letters: A, C, G, and T, which denote adenosine, cytidine, guanosine, and thymidine, respectively), the nucleotides are presented in the 5′ to 3′ order from the left to the right. In certain embodiments, the sequence of an oligonucleotide of the present invention contains the letter M. As used herein, the letter “M” represents a degenerative base, which can be A or C with substantially equal probability. Thus, for example, in the context of the present invention, if an oligonucleotide contains one degenerative base M, the oligonucleotide is a substantially equimolar mixture of two subpopulations of a first oligonucleotide where the degenerative base is A and a second oligonucleotide where the degenerative base is C, the first and second oligonucleotide being otherwise substantially identical.

The term “3′” refers to a region or position in a polynucleotide or oligonucleotide 3′ (i.e., downstream) from another region or position in the same polynucleotide or oligonucleotide. The term “5′” refers to a region or position in a polynucleotide or oligonucleotide 5′ (i.e., upstream) from another region or position in the same polynucleotide or oligonucleotide. The terms “3′ end” and “3′ terminus”, as used herein in reference to a nucleic acid molecule, refer to the end of the nucleic acid which contains a free hydroxyl group attached to the 3′ carbon of the terminal pentose sugar. The term “5′ end” and “5′ terminus”, as used herein in reference to a nucleic acid molecule, refers to the end of the nucleic acid molecule which contains a free hydroxyl or phosphate group attached to the 5′ carbon of the terminal pentose sugar.

The term “isolated”, when referring to an oligonucleotide means an oligonucleotide, which by virtue of its origin or manipulation, is separated from at least some of the components with which it is naturally associated. By “isolated”, it is alternatively or additionally meant that the oligonucleotide of interest is produced or synthesized by the hand of man.

The term “active fragment”, as used herein in reference to an oligonucleotide (e.g., an oligonucleotide sequence provided herein), refers to any nucleic acid molecule comprising a nucleotide sequence sufficiently homologous to or derived from the nucleotide sequence of the oligonucleotide, which includes fewer nucleotides than the full length oligonucleotide, and retains at least one biological property of the entire sequence. Typically, active fragments comprise a sequence with at least one activity of the full length oligonucleotide. An active fragment or portion of an oligonucleotide sequence of the present invention can be a nucleic acid molecule which is, for example, about 10, about 15, about 20, about 25, about 30 or more than 30 oligonucleotides in length and can be used as amplification primer and/or detection probe for the detection of HBV in a biological sample.

The term “sufficiently homologous”, when used herein in reference to an active fragment of an oligonucleotide, refers to a nucleic acid molecule that has a sequence homology of at least about 35% compared to the oligonucleotide. In certain embodiments, the sequence homology is at least about 40%, at least about 45%, at least about 50%, at least about 55%, at least about 60%, at least about 65%, at least about 70%, at least about 75%, at least about 80%, at least about 85%, at least about 90%, at least about 95% or more.

The terms “homology” and “identity” are used herein interchangeably, and refer to the sequence similarity between two nucleic acid molecules. Calculation of the percent homology or identity of two nucleic acid sequences, can be performed by aligning the two sequences for optimal comparison purposes (e.g., gaps can be introduced in one or both of a first and a second nucleic acid sequences for optimal alignment and non-homologous sequences can be disregarded for comparison purposes). In certain embodiments, the length of a sequence aligned for comparison purposes is at least about 30%, at least about 40%, at least about 50%, at least about 60%, at least about 70%, at least about 80%, at least about 90%, at least about 95% or 100% of the length of the reference sequence. The nucleotides at corresponding nucleotide positions are then compared. When a position in the first sequence is occupied by the same nucleotide as the corresponding position in the second sequence, then the molecules are identical (or homologous) at that position. The percent identity between the two sequences is a function of the number of identical positions shared by the sequences, taking into account the number of gaps, and the length of each gap, which needs to be introduced for optimal alignment of the two sequences.

The comparison of sequences and determination of percent identity between two sequences can be accomplished using a mathematical algorithm. For example, the percent identity between two nucleotide sequences can be determined using the algorithm of Meyers and Miller (CABIOS, 1989, 4: 11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package (available at http://www.gcg.com), using a NWSgapdna.CMP matrix.

The term “hybridization”, as used herein, refers to the formation of complexes (also called duplexes or hybrids) between nucleotide sequences which are sufficiently complementary to form complexes via Watson-Crick base pairing or non-canonical base pairing. It will be appreciated that hybridizing sequences need not have perfect complementarity to provide stable hybrids. In many situations, stable hybrids will form where fewer than about 10% of the bases are mismatches. Accordingly, as used herein, the term “complementary” refers to a nucleic acid molecule that forms a stable duplex with its complement under assay conditions, generally where there is about 90% or greater homology. Those skilled in the art understand how to estimate and adjust the stringency of hybridization conditions such that sequences having at least a desired level of complementarity will stably hybridize, while those having lower complementarity will not. For examples of hybridization conditions and parameters see, e.g., J. Sambrook et al., “Molecular Cloning: A Laboratory Manual”, 1989, Second Edition, Cold Spring Harbor Press: Plainview, N.Y.; F. M. Ausubel, “Current Protocols in Molecular Biology”, 1994, John Wiley & Sons: Secaucus, N.J. Complementarity between two nucleic acid molecules is said to be “complete”, “total” or “perfect” if all the nucleic acids' bases are matched, and is said to be “partial” otherwise.

As used herein, the term “amplification” refers to a method/process that increases the representation of a population of a specific nucleic acid sequence in a sample by producing multiple (i.e., at least 2) copies of the desired sequence. Methods for nucleic acid amplification are known in the art and include, but are not limited to, polymerase chain reaction (PCR) and ligase chain reaction (LCR). In a typical PCR amplification reaction, a nucleic acid sequence of interest is often amplified at least fifty thousand fold in amount over its amount in the starting sample. A “copy” or “amplicon” does not necessarily mean perfect sequence complementarity or identity to template sequence. For example, copies can include nucleotide analogs such as deoxyinosine, intentional sequence alterations (such as sequence alterations introduced through a primer comprising a sequence that is hybridizable but not complementary to the template), and/or sequence errors that occur during amplification. Amplification methods (such as polymerase chain reaction or PCR) are known in the art and are discussed in more detail below.

As used herein, the term “target sequence” refers to a particular nucleic acid sequence which is to be detected and/or amplified. Preferably, target sequences include nucleic acid sequences to which the primers complex in a PCR reaction. Target sequences may also include a probe hybridizing region with which a detection probe will form a stable hybrid under desired conditions. As will be recognized by one of ordinary skill in the art, a target sequence may be single-stranded or double-stranded. In the context of the present invention, target sequences of interest are within the HBV surface antigen gene (region encompassing nucleotides 1560 to 2240).

The term “primer” and “amplification primer” are used herein interchangeably. They refer to an oligonucleotide which acts as a point of initiation of synthesis of a primer extension product, when placed under suitable conditions (e.g., buffer, salt, temperature and pH), in the presence of nucleotides and an agent for nucleic acid polymerization (e.g., a DNA-dependent or RNA-dependent polymerase). The primer is preferably single-stranded for maximum efficiency in amplification, but may alternatively be double-stranded. If double-stranded, the primer may first be treated (e.g., denatured) to allow separation of its strands before being used to prepare extension products. Such a denaturation step is typically performed using heat, but may alternatively be carried out using alkali, followed by neutralization. A typical primer comprises about 10 to about 35 nucleotides in length of a sequence substantially complementary to the target sequence. However, a primer can also contain additional sequences. For example, amplification primers used in Strand Displacement Amplification (SDA) preferably include a restriction endonuclease recognition at site 5′ to the target binding sequence (see, for example, U.S. Pat. Nos. 5,270,184 and 5,455,166). Nucleic Acid Sequence Based Amplification (NASBA), Self Sustaining Sequence Replication (3SR), and Transcription-Medicated Amplification (TMA) primers preferably include an RNA polymerase promoter linked to the target binding sequence of the primer. Methods for linking such specialized sequences to a binding target sequence for use in a selected amplification reaction are well-known in the art.

The terms “forward primer” and “forward amplification primer” are used herein interchangeably, and refer to a primer that hybridizes (or anneals) to the target sequence (template strand). The terms “reverse primer” and “reverse amplification primer” are used herein interchangeably, and refer to a primer that hybridizes (or anneals) to the complementary target strand 3′ with respect to the forward primer.

The term “amplification conditions”, as used herein, refers to conditions that promote annealing and/or extension of primer sequences. Such conditions are well-known in the art and depend on the amplification method selected. Thus, for example, in a PCR reaction, amplification conditions generally comprise thermal cycling, i.e., cycling of the reaction mixture between two or more temperatures. In isothermal amplification reactions, amplification occurs without thermal cycling although an initial temperature increase may be required to initiate the reaction. Amplification conditions encompass all reaction conditions including, but not limited to, temperature and temperature cycling, buffer, salt, ionic strength, pH, and the like.

As used herein, the term “amplification reaction reagents” refers to reagents used in nucleic acid amplification reactions and may include, but are not limited to, buffers, enzymes having reverse transcriptase and/or polymerase activity or exonuclease activity; enzyme cofactors such as magnesium or manganese; salts; nicotinamide adenine dinuclease NAD; and deoxynucleoside triphosphates (dNTPs) such as deoxyadenosine triphosphate, deoxyguanosine triphosphate, deoxycytidine triphosphate, and thymidine triphosphate. Amplification reaction reagents may readily be selected by one skilled in the art depending on the amplification method used.

The terms “probe” and “detection probe” are used herein interchangeably and refer to an oligonucleotide capable of selectively hybridizing to a portion of a target sequence under appropriate conditions. In certain embodiments, a detection probe is labeled with a detectable moiety.

The terms “labeled” and “labeled with a detectable agent (or moiety)” are used herein interchangeably to specify that an entity (e.g., an oligonucleotide detection probe) can be visualized, for example following binding to another entity (e.g., an amplification reaction product or amplicon). Preferably, the detectable agent or moiety is selected such that it generates a signal which can be measured and whose intensity is related to (e.g., proportional to) the amount of bound entity. A wide variety of systems for labeling and/or detecting nucleic acid molecules are well-known in the art. Labeled nucleic acids can be prepared by incorporation of, or conjugation to, a label that is directly or indirectly detectable by spectroscopic, photochemical, biochemical, immunochemical, electrical, optical, or chemical means. Suitable detectable agents include, but are not limited to, radionuclides, fluorophores, chemiluminescent agents, microparticles, enzymes, colorimetric labels, magnetic labels, haptens, Molecular Beacons, and aptamer beacons.

The terms “fluorophore”, “fluorescent moiety”, and “fluorescent dye” are used herein interchangeably. They refer to a molecule that absorbs a quantum of electromagnetic radiation at one wavelength, and emits one or more photons at a different, typically longer, wavelength in response. Numerous fluorescent dyes of a wide variety of structures and characteristics are suitable for use in the practice of the present invention. Methods and materials are known for fluorescently labeling nucleic acid molecules (see, for example, R. P. Haugland, “Molecular Probes: Handbook of Fluorescent Probes and Research Chemicals 1992-1994”, 5^{th} Ed., 1994, Molecular Probes, Inc.). Preferably, a fluorescent moiety absorbs and emits light with high efficiency (i.e., it has a high molar absorption coefficient at the excitation wavelength used, and a high fluorescence quantum yield), and is photostable (i.e., it does not undergo significant degradation upon light excitation within the time necessary to perform the analysis). Rather than being directly detectable themselves, some fluorescent molecules transfer energy to another fluorescent molecule in a process of fluorescent resonance energy transfer (FRET), and the second fluorescent molecule produces the detectable signal. Such FRET fluorescent dye pairs are also encompassed by the term “fluorescent moiety”. The use of physically linked fluorescent reporter/quencher moiety is also within the scope of the invention. In such embodiments, when the fluorescent reporter and quenching moiety are held in close proximity, such as at the ends of a nucleic acid probe, the quenching moiety prevents detection of a fluorescent signal from the reporter moiety. When the two moieties are physically separated, such as, for example, after cleavage by a Taq DNA polymerase, the fluorescent signal from the reporter moiety becomes detectable.

The term “directly detectable”, when used herein in reference to a label, or detectable moiety, means that the label does not require further reaction or manipulation to be detectable. For example, a fluorescent moiety is directly detectable by fluorescence spectroscopy methods. The term “indirectly detectable”, when used herein in reference to a label, or detectable moiety, means that the label becomes detectable after further reaction or manipulation. For example, a hapten becomes detectable after reaction with an appropriate antibody attached to a reporter, such as a fluorescent dye.

As used herein, the term “viral load” has its art understood meaning and refers to a measure of the persistence or severity of a viral infection. Viral load can be estimated by calculating the amount of virus in an involved body fluid (e.g., a viral load may be given in nucleic acid copies or international units per milliliter of blood).

As used herein, the term “diagnosis” refers to a process aimed at determining if an individual is afflicted with a disease or ailment. In the context of the present invention, the term “HBV diagnosis” refers to a process aimed at one or more of: determining if an individual is infected with HBV (i.e., if HBV nucleic acids are present in a biological sample obtained from the individual), determining the HBV viral load of an individual (e.g., in blood, serum or plasma or other biological fluid or tissue), determining if an individual is afflicted with a HBV disease, determining the severity of the HBV disease, determining the progression of the HBV disease, determining the likelihood of developing a HBV disease, determining recurrence of HBV after a patient's immune system has been compromised (e.g., after organ transplantation), determining response of HBV in settings of viral co-infection (e.g., HIV and/or HCV) and treatment with cross-therapeutic drugs (such as, e.g., 3TC), and determining the efficacy of a HBV therapy undergone by the individual.

The terms “normal” and “healthy” are used herein interchangeably. They refer to an individual or group of individuals who are not infected with HBV. Preferably, a normal individual is also not suffering from a disease, such as cirrhosis or another liver disease, which can be associated with HBV infection.

In the context of the present invention, the terms “control sample” and “reference sample” are used herein interchangeably and refer to one or more biological samples isolated from an individual or group of individuals that are classified as normal. A control or reference sample can also refer to a biological sample isolated from a patient or group of patients diagnosed with a specific HBV disease or with a specific stage of a HBV disease or with a specific genotype.

The term “treatment” is used herein to characterize a method that is aimed at (1) delaying or preventing the onset of a HBV disease; (2) slowing down or stopping the progression, aggravation, or deterioration of the symptoms of the disease; (3) bringing about ameliorations of the symptoms of the disease; and/or (4) curing the disease. A treatment may be administered prior to the onset of the disease, for a prophylactic or preventive action, or may be administered after initiation of the disease, for a therapeutic action.

### DETAILED DESCRIPTION OF CERTAIN PREFERRED EMBODIMENTS

As mentioned above, the present invention relates to methods and reagents for specifically and selectively detecting HBV in biological samples. More specifically, the inventive methods use HBV-specific oligonucleotide sequences and sensitive nucleic acid amplification-based techniques. In certain embodiments, the HBV-specific oligonucleotide sequences recognize all eight genotypes of HBV and the inventive methods allow detection of a very wide range of copy numbers of HBV in biological samples.

### I—Oligonucleotide Sequences for Amplification Primers and Detection Probes

### Inventive Oligonucleotide Sequences

In one aspect, the present invention provides oligonucleotide sequences that can be used in nucleic acid amplification tests for the specific detection of target sequences within the HBV surface antigen gene.

The genome of HBV is a 3.2 kilobase circular partially double-stranded DNA containing four overlapping open reading frames (C-, S-, P- and X-ORFs) encoding surface, pre-core, core, polymerase, and X genes (see, for example, F. Galibert et al., Nature, 1979, 281: 646-650; H. Okamoto et al., J. Gen. Virol., 1986, 67: 2305-2314; Y. Ono et al., Nucl. Acids Res., 1983, 11: 1747-1757; H. Okamoto et al., J. Gen. Virology, 1988, 69: 2575-2583). The C-ORF codes for the core antigen (HBcAg) and for a pre-core protein which is co-translationally processed and secreted as HBeAg; the S-ORF codes for the surface antigen (HBsAg); the P-ORF codes for the viral polymerase (pol) that has RNA- and DNA-dependent DNA polymerase and RNase H activities; and the X-ORF codes for a protein with trans-activating activity.

As mentioned above, HBsAg is conventionally classified into four serological subtypes, adw, adr, ayw and ayr (A. M. Courouce et al., J. Dev. Biol. Stand., 1982, 54: 527-534). Recently, eight HBV genotypes A-H, have been classified based primarily on an inter-genotype divergence of >8% (P. Arauz-Ruiz et al., J. Gen. Virol., 2002, 83: 2059-2073; H. Norder et al., Virol., 1994, 198, 489-503; H. Okamoto et al., J. Gen. Virol., 1988, 69: 2575-2583; L. Stuyer et al., J. Gen. Virol., 2000, 81: 67-74). The correlation between serologic subtypes and genotypes has been partially established (H. Norder et al., Intervirology, 2004, 47: 289-309). The prevalence of different genotypes varies geographically and is strongly associated with ethnicity. However, intertypic recombinations between different HBV genotypes have been observed (C. Cui et al., J. Gen. Virol., 2002, 83: 2773-2777; C. Hannoun et al., J. Gen. Virol., 2005, 86: 2047-2056; V. Morozov et al., Gene, 2000, 260: 55-65). The concept that HBV genotypes may influence the course of HBV disease and the prognosis of treatment has now been recognized (S. Schaefer, J. Viral Hepat., 2005, 12: 111-124). Therefore, diagnostic procedures that do not take into account the variability of HBV risk generating false negatives and leading to inaccurately low quantitative results. Additionally or alternatively, genetic mutations in the gene encoding HBsAg may result in loss of detection by conventional assays, leading to false negatives. In certain embodiments, methods of the present invention are useful in detecting HBV which has undergone one or more genetic mutations in the gene encoding HbsAg, leading to elimination or reduction in false negatives. Additionally or alternatively, in certain embodiments, methods of the present invention are useful in detecting HBV which has undergone one or more genetic mutations in other genes, including but not limited to, the pre-core, core, polymerase, and X genes.

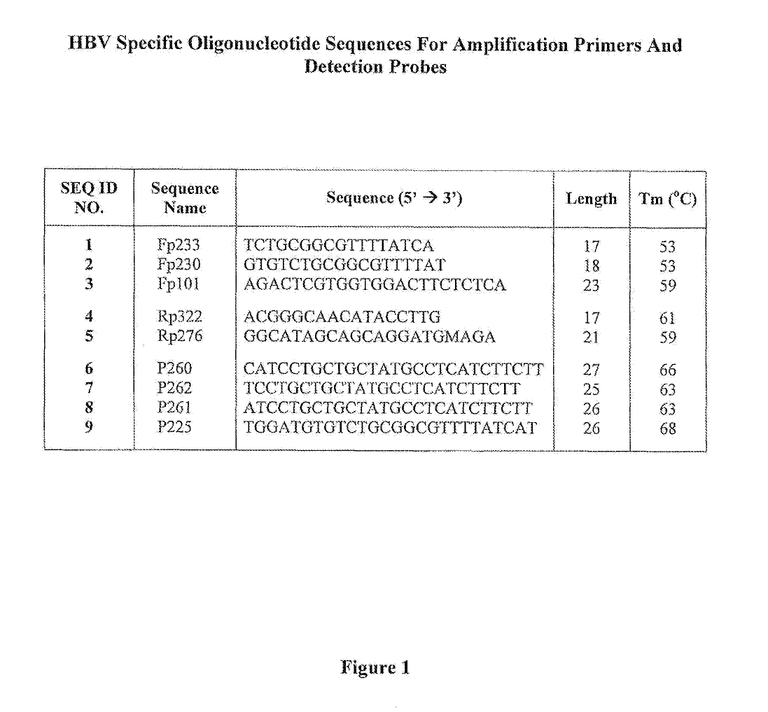

Accordingly, the present invention provides oligonucleotide sequences for amplification primers and detection probes which recognize more than one genotype of HBV. In certain embodiments, the inventive oligonucleotide sequences recognize all eight genotypes of HBV. In certain embodiments, the inventive oligonucleotide sequences recognize HBV which has undergone one or more genetic mutations. Exemplary oligonucleotide sequences of the present invention are presented in the table presented on FIG. 1 (SEQ. ID NOs. 1 to 9). These sequences were derived from the HBV surface antigen gene and identified by the present Applicants by sequence alignment of 145 HBV DNA sequences (representing all eight genotypes) deposited in GenBank using the software program Vector NTI Advance™ (Invitrogen) and designed using the software program Primer Express® (Applied Biosystems). The selected sequences were highly conserved for all HBV genotypes. These sequences were tested using BLAST search to verify specificity for HBV and absence of cross-reactivity with human genomic DNA. It is to be understood that the invention also encompasses the complementary sequences of SEQ. ID NOs. 1 to 9, and any active fragments thereof.

As will be appreciated by one skilled in the art, any of the oligonucleotide sequences (or active fragment thereof) disclosed herein for amplification, detection or quantitation of HBV may be employed either as detection probe or amplification primer, depending on the intended use or assay format. For example, an inventive oligonucleotide sequence used as an amplification primer in one assay may be used as a detection probe in another assay. A given sequence may be modified, for example, by attaching to the inventive oligonucleotide sequence, a specialized sequence (e.g., a promoter sequence) required by the selected amplification method, or by attaching a fluorescent dye to facilitate detection. It is also to be understood that an oligonucleotide according to the present invention may include one or more sequences which serve as spacers, linkers, sequences for labeling or binding to an enzyme, which may impart added stability or susceptibility to degradation process or other desirable property to the oligonucleotide.

Based on the oligonucleotide sequences provided by the present invention, one or more oligonucleotide analogues can be prepared (see below). Such analogues may contain alternative structures such as peptide nucleic acids or “PNAs” (i.e., molecules with a peptide-like backbone instead of the phosphate sugar backbone of naturally-occurring nucleic acids) and the like. These alternative structures, representing the sequences of the present invention, are likewise part of the present invention. Similarly, it is understood that oligonucleotides comprising sequences of the present invention may contain deletions, additions, and/or substitutions of nucleic acid bases, to the extent that such alterations do not negatively affect the properties of the nucleic acid molecules. In particular, such alterations should not result in significant lowering of the hybridizing properties of the oligonucleotides.

### Primer Sets and Primer/Probe Sets

In another aspect, the present invention relates to combinations of oligonucleotide sequences disclosed herein for the detection of HBV in biological samples. More specifically, the present invention provides primer sets and primer/probe sets for HBV amplification and detection.

As used herein, the term “primer set” refers to two or more primers which together can be used to prime the amplification of a nucleotide sequence of interest (e.g., a target sequence within the HBV surface antigen gene). In certain embodiments, the term “primer set” refers to a forward primer and a reverse primer. Such primer sets or primer pairs are particularly useful in PCR amplification reactions.

Examples of primer sets comprising a forward amplification primer and a reverse amplification primer include:

Primer Set 1, which comprises a forward primer having a sequence as set forth in SEQ ID NO. 1 (5′-TCTGCGGCGTTTTATCA-3′) or any active fragment thereof, and a reverse primer having a sequence as set forth in SEQ ID NO. 4 (5′-ACGGGCAACATACCTTG-3′) or any active fragment thereof;

Primer Set 2, which comprises a forward primer having a sequence as set forth in SEQ ID NO. 2 (5′-GTGTCTGCGGCGTTTTAT-3′) or any active fragment thereof, and a reverse primer having a sequence as set forth in SEQ ID NO. 4 (5′-ACGGGCAACATACCTTG-3′) or any active fragment thereof; and

Primer Set 3, which comprises a forward primer having a sequence as set forth in SEQ ID NO. 3 (5′-AGACTCGTGGTGGACTTCTCTCA-3′) or any active fragment thereof, and a reverse primer having a sequence as set forth in SEQ ID NO. 5 (5′-GGCATAGCAGCAGGATGMAGA-3′) or any active fragment thereof.

These primer sets can be used according to any nucleic acid amplification technique that employs one or more oligonucleotides to amplify a target sequence (as discussed below). Amplification products produced using inventive primer sets of the present invention may be detected using a variety of detection methods well known in the art. For example, amplification products may be detected using agarose gel electrophoresis and visualization by ethidium bromide staining and exposure to ultraviolet (UV) light or by sequence analysis of the amplification product for confirmation of HBV identity.

Alternatively, probe sequences can be employed using a variety of homogeneous or heterogeneous methodologies to detect amplification products. Generally in all such methods, the probe hybridizes to a strand of an amplification product (or amplicon) to form an amplification product/probe hybrid. The hybrid can then be directly or indirectly detected, for example using labels on the primers, probes or both the primers and probes.

Accordingly, the present invention provides primer/probe sets that can be used with nucleic acid amplification procedures to specifically amplify and detect HBV target sequences in test samples. As used herein, the term “primer/probe set” refers to a combination comprising two or more primers which together are capable of priming the amplification of a nucleotide sequence of interest (e.g., a target sequence within the HVB surface antigen gene), and at least one probe which can detect the amplified nucleotide sequence. Generally, the probe hybridizes to a strand of the amplification product (amplicon) to form a detectable amplification product/probe hybrid.

Certain inventive primer/probe sets comprise a primer set, as described above, and at least one detection probe. The detection probe may comprise a detectable moiety. In certain embodiments, the detection probe comprises a fluorescent reporter moiety attached at the 5′ end and a quencher moiety attached at the 3′ end.

Examples of primer/probe sets provided by the present invention include:

Primer/Probe Set 1(a), which comprises a forward primer having a sequence as set forth in SEQ ID NO. 1 (5′-TCTGCGGCGTTTTATCA-3′) or any active fragment thereof, a reverse primer having a sequence as set forth in SEQ ID NO. 4 (5′-ACGGGCAACATACCTTG-3 ‘) or any active fragment thereof, and a detection probe having a sequence as set forth in SEQ ID NO. 6 (5’-CATCCTGCTGCTATGCCTCATCTTCTT-3′) or any active fragment thereof;

Primer/Probe Set 1(b), which comprises a forward primer having a sequence as set forth in SEQ ID NO. 1 (5′-TCTGCGGCGTTTTATCA-3′) or any active fragment thereof, a reverse primer having a sequence as set forth in SEQ ID NO. 4 (5′-ACGGGCAACATACCTTG-3′) or any active fragment thereof, and a detection probe having a sequence as set forth in SEQ ID NO. 7 (5′-TCCTGCTGCTATGCCTCATCTTCTT-3′) or any active fragment thereof;

Primer/Probe Set 1(c), which comprises a forward primer having a sequence as set forth in SEQ ID NO. 1 (5′-TCTGCGGCGTTTTATCA-3′) or any active fragment thereof, a reverse primer having a sequence as set forth in SEQ ID NO. 4 (5′-ACGGGCAACATACCTTG-3′) or any active fragment thereof, and a detection probe having a sequence as set forth in SEQ ID NO. 8 (5′-ATCCTGCTGCTATGCCTCATCTTCTT-3′) or any active fragment thereof;

Primer/Probe Set 2(a), which comprises a forward primer having a sequence as set forth in SEQ ID NO. 2 (5′-GTGTCTGCGGCGTTTTAT-3′) or any active fragment thereof, a reverse primer having a sequence as set forth in SEQ ID NO. 4 (5′-ACGGGCAACATACCTTG-3′) or any active fragment thereof, and a detection probe having a sequence as set forth in SEQ ID NO. 6 (5′-CATCCTGCTGCTATGCCTCATCTTCTT-3′) or any active fragment thereof;

Primer/Probe Set 2(b), which comprises a forward primer having a sequence as set forth in SEQ ID NO. 2 (5′-GTGTCTGCGGCGTTTTAT-3′) or any active fragment thereof, a reverse primer having a sequence as set forth in SEQ ID NO. 4 (5′-ACGGGCAACATACCTTG-3′) or any active fragment thereof, and a detection probe having a sequence as set forth in SEQ ID NO. 7 (5′-TCCTGCTGCTATGCCTCATCTTCTT-3′) or any active fragment thereof;

Primer/Probe Set 2(c), which comprises a forward primer having a sequence as set forth in SEQ ID NO. 2 (5′-GTGTCTGCGGCGTTTTAT-3′) or any active fragment thereof, a reverse primer having a sequence as set forth in SEQ ID NO. 4 (5′-ACGGGCAACATACCTTG-3′) or any active fragment thereof, and a detection probe having a sequence as set forth in SEQ ID NO. 8 (5′-ATCCTGCTGCTATGCCTCATCTTCTT-3′) or any active fragment thereof; and

Primer/Probe Set 3, which comprises a forward primer having a sequence as set forth in SEQ ID NO. 3 (5′-AGACTCGTGGTGGACTTCTCTCA-3′) or any active fragment thereof, a reverse primer having a sequence as set forth in SEQ ID NO. 5 (5′-GGCATAGCAGCAGGATGMAGA-3′) or any active fragment thereof, and a detection probe having a sequence as set forth in SEQ ID NO. 9 (5′-TGGATGTGTCTGCGGCGTTTTATCAT-3′) or any active fragment thereof.

### Oligonucleotide Preparation

Oligonucleotides of the invention may be prepared by any of a variety of methods (see, for example, J. Sambrook et al., “Molecular Cloning: A Laboratory Manual”, 1989, 2^{nd} Ed., Cold Spring Harbour Laboratory Press: New York, N.Y.; “PCR Protocols: A Guide to Methods and Applications”, 1990, M. A. Innis (Ed.), Academic Press: New York, N.Y.; P. Tijssen “Hybridization with Nucleic Acid Probes—Laboratory Techniques in Biochemistry and Molecular Biology (Parts I and II)”, 1993, Elsevier Science; “PCR Strategies”, 1995, M. A. Innis (Ed.), Academic Press: New York, N.Y.; and “Short Protocols in Molecular Biology”, 2002, F. M. Ausubel (Ed.), 5^{th} Ed., John Wiley & Sons: Secaucus, N.J.). For example, the oligonucleotides may be prepared using any of a variety of chemical techniques well-known in the art, including, for example, chemical synthesis and polymerization based on a template as described, for example, in S. A. Narang et al., Meth. Enzymol. 1979, 68: 90-98; E. L. Brown et al., Meth. Enzymol. 1979, 68: 109-151; E. S. Belousov et al., Nucleic Acids Res. 1997, 25: 3440-3444; D. Guschin et al., Anal. Biochem. 1997, 250: 203-211; M. J. Blommers et al., Biochemistry, 1994, 33: 7886-7896; and K. Frenkel et al., Free Radic. Biol. Med. 1995, 19: 373-380; and U.S. Pat. No. 4,458,066).

For example, oligonucleotides may be prepared using an automated, solid-phase procedure based on the phosphoramidite approach. In such a method, each nucleotide is individually added to the 5′-end of the growing oligonucleotide chain, which is attached at the 3′-end to a solid support. The added nucleotides are in the form of trivalent 3′-phosphoramidites that are protected from polymerization by a dimethoxytriyl (or DMT) group at the 5′ position. After base-induced phosphoramidite coupling, mild oxidation to give a pentavalent phosphotriester intermediate and DMT removal provides a new site for oligonucleotide elongation. The oligonucleotides are then cleaved off the solid support, and the phosphodiester and exocyclic amino groups are deprotected with ammonium hydroxide. These syntheses may be performed on oligo synthesizers such as those commercially available from Perkin Elmer/Applied Biosystems, Inc. (Foster City, Calif.), DuPont (Wilmington, Del.) or Milligen (Bedford, Mass.). Alternatively, oligonucleotides can be custom made and ordered from a variety of commercial sources well-known in the art, including, for example, the Midland Certified Reagent Company (Midland, Tex.), ExpressGen, Inc. (Chicago, Ill.), Operon Technologies, Inc. (Huntsville, Ala.), and many others.

Purification of oligonucleotides of the invention, where necessary or desired, may be carried out by any of a variety of methods well-known in the art. Purification of oligonucleotides is typically performed either by native acrylamide gel electrophoresis, by anion-exchange HPLC as described, for example, by J. D. Pearson and F. E. Regnier (J. Chrom., 1983, 255: 137-149) or by reverse phase HPLC (G. D. McFarland and P. N. Borer, Nucleic Acids Res., 1979, 7: 1067-1080).

The sequence of oligonucleotides can be verified using any suitable sequencing method including, but not limited to, chemical degradation (A. M. Maxam and W. Gilbert, Methods of Enzymology, 1980, 65: 499-560), matrix-assisted laser desorption ionization time-of-flight (MALDI-TOF) mass spectrometry (U. Pieles et al., Nucleic Acids Res., 1993, 21: 3191-3196), mass spectrometry following a combination of alkaline phosphatase and exonuclease digestions (H. Wu and H. Aboleneen, Anal. Biochem., 2001, 290: 347-352), and the like.

As already mentioned above, modified oligonucleotides may be prepared using any of several means known in the art. Non-limiting examples of such modifications include methylation, “caps”, substitution of one or more of the naturally-occurring nucleotides with a nucleotide analog, and internucleotide modifications such as, for example, those with uncharged linkages (e.g., methyl phosphonates, phosphotriesters, phosphoroamidates, carbamates, etc), or charged linkages (e.g., phosphorothioates, phosphorodithioates, etc). Oligonucleotides may contain one or more additional covalently linked moieties, such as, for example, proteins (e.g., nucleases, toxins, antibodies, signal peptides, poly-L-lysine, etc), intercalators (e.g., acridine, psoralen, etc), chelators (e.g., metals, radioactive metals, iron, oxidative metals, etc), and alkylators. Oligonucleotides may also be derivatized by formation of a methyl or ethyl phosphotriester or an alkyl phosphoramidate linkage. Furthermore, the oligonucleotide sequences of the present invention may also be modified with a label.

### Labeling of Oligonucleotide Sequences

In certain embodiments, the detection probes or amplification primers or both probes and primers are labeled with a detectable agent or moiety before being used in amplification/detection assays. In certain preferred embodiments, the detection probes are labeled with a detectable agent. The role of a detectable agent is to allow visualization and facilitate detection of amplified target sequences. Preferably, the detectable agent is selected such that it generates a signal which can be measured and whose intensity is related (e.g., proportional) to the amount of amplification products in the sample being analyzed.

The association between the oligonucleotide and detectable agent can be covalent or non-covalent. Labeled detection probes can be prepared by incorporation of or conjugation to a detectable moiety. Labels can be attached directly to the nucleic acid sequence or indirectly (e.g., through a linker). Linkers or spacer arms of various lengths are known in the art and are commercially available, and can be selected to reduce steric hindrance, or to confer other useful or desired properties to the resulting labeled molecules (see, for example, E. S. Mansfield et al., Mol. Cell. Probes, 1995, 9: 145-156).

Methods for labeling nucleic acid molecules are well-known in the art. For a review of labeling protocols, label detection techniques, and recent developments in the field, see, for example, L. J. Kricka, Ann. Clin. Biochem. 2002, 39: 114-129; R. P. van Gijlswijk et al., Expert Rev. Mol. Diagn. 2001, 1: 81-91; and S. Joos et al., J. Biotechnol. 1994, 35: 135-153. Standard nucleic acid labeling methods include: incorporation of radioactive agents, direct attachments of fluorescent dyes (L. M. Smith et al., Nucl. Acids Res., 1985, 13: 2399-2412) or of enzymes (B. A. Connoly and O. Rider, Nucl. Acids. Res., 1985, 13: 4485-4502); chemical modifications of nucleic acid molecules making them detectable immunochemically or by other affinity reactions (T. R. Broker et al., Nucl. Acids Res. 1978, 5: 363-384; E. A. Bayer et al., Methods of Biochem. Analysis, 1980, 26: 1-45; R. Langer et al., Proc. Natl. Acad. Sci. USA, 1981, 78: 6633-6637; R. W. Richardson et al., Nucl. Acids Res. 1983, 11: 6167-6184; D. J. Brigati et al., Virol. 1983, 126: 32-50; P. Tchen et al., Proc. Natl. Acad. Sci. USA, 1984, 81: 3466-3470; J. E. Landegent et al., Exp. Cell Res. 1984, 15: 61-72; and A. H. Hopman et al., Exp. Cell Res. 1987, 169: 357-368); and enzyme-mediated labeling methods, such as random priming, nick translation, PCR and tailing with terminal transferase (for a review on enzymatic labeling, see, for example, J. Temsamani and S. Agrawal, Mol. Biotechnol. 1996, 5: 223-232). More recently developed nucleic acid labeling systems include, but are not limited to: ULS (Universal Linkage System), which is based on the reaction of monoreactive cisplatin derivatives with the N7 position of guanine moieties in DNA (R. J. Heetebrij et al., Cytogenet. Cell. Genet. 1999, 87: 47-52), psoralen-biotin, which intercalates into nucleic acids and upon UV irradiation becomes covalently bonded to the nucleotide bases (C. Levenson et al., Methods Enzymol. 1990, 184: 577-583; and C. Pfannschmidt et al., Nucleic Acids Res. 1996, 24: 1702-1709), photoreactive azido derivatives (C. Neves et al., Bioconjugate Chem. 2000, 11: 51-55), and DNA alkylating agents (M. G. Sebestyen et al., Nat. Biotechnol. 1998, 16: 568-576).

Any of a wide variety of detectable agents can be used in the practice of the present invention. Suitable detectable agents include, but are not limited to, various ligands, radionuclides (such as, for example, ³²P, ³⁵S, ³H, ¹⁴C, ¹²⁵I, ¹³¹I, and the like); fluorescent dyes (for specific exemplary fluorescent dyes, see below); chemiluminescent agents (such as, for example, acridinium esters, stabilized dioxetanes, and the like); spectrally resolvable inorganic fluorescent semiconductor nanocrystals (i.e., quantum dots), metal nanoparticles (e.g., gold, silver, copper and platinum) or nanoclusters; enzymes (such as, for example, those used in an ELISA, e.g., horseradish peroxidase, beta-galactosidase, luciferase, alkaline phosphatase); colorimetric labels (such as, for example, dyes, colloidal gold, and the like); magnetic labels (such as, for example, Dynabeads™); and biotin, dioxigenin or other haptens and proteins for which antisera or monoclonal antibodies are available.

In certain preferred embodiments, the inventive detection probes are fluorescently labeled. Numerous known fluorescent labeling moieties of a wide variety of chemical structures and physical characteristics are suitable for use in the practice of this invention. Suitable fluorescent dyes include, but are not limited to, fluorescein and fluorescein dyes (e.g., fluorescein isothiocyanine or FITC, naphthofluorescein, 4′,5′-dichloro-2′,7′-dimethoxy-fluorescein, 6-carboxyfluorescein or FAM), carbocyanine, merocyanine, styryl dyes, oxonol dyes, phycoerythrin, erythrosin, eosin, rhodamine dyes (e.g., carboxytetramethylrhodamine or TAMRA, carboxyrhodamine 6G, carboxy-X-rhodamine (ROX), lissamine rhodamine B, rhodamine 6G, rhodamine Green, rhodamine Red, tetramethylrhodamine or TMR), coumarin and coumarin dyes (e.g., methoxycoumarin, dialkylaminocoumarin, hydroxycoumarin and aminomethylcoumarin or AMCA), Oregon Green Dyes (e.g., Oregon Green 488, Oregon Green 500, Oregon Green 514), Texas Red, Texas Red-X, Spectrum Red™, Spectrum Green™, cyanine dyes (e.g., Cy-3™, Cy-5™, Cy-3.5™, Cy-5.5™), Alexa Fluor dyes (e.g., Alexa Fluor 350, Alexa Fluor 488, Alexa Fluor 532, Alexa Fluor 546, Alexa Fluor 568, Alexa Fluor 594, Alexa Fluor 633, Alexa Fluor 660 and Alexa Fluor 680), BODIPY dyes (e.g., BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665), IRDyes (e.g., IRD40, IRD 700, IRD 800), and the like. For more examples of suitable fluorescent dyes and methods for linking or incorporating fluorescent dyes to nucleic acid molecules see, for example, “The Handbook of Fluorescent Probes and Research Products”, 9^{th} Ed., Molecular Probes, Inc., Eugene, Oreg. Fluorescent dyes as well as labeling kits are commercially available from, for example, Amersham Biosciences, Inc. (Piscataway, N.J.), Molecular Probes Inc. (Eugene, Oreg.), and New England Biolabs Inc. (Berverly, Mass.).

Rather than being directly detectable themselves, some fluorescent groups (donors) transfer energy to another fluorescent group (acceptor) in a process of fluorescent resonance energy transfer (FRET) in which the second group produces the detected fluorescent signal. In these systems, the oligonucleotide detection probe may, for example, become detectable when hybridized to an amplified target sequence. Examples of FRET acceptor/donor pairs suitable for use in the present invention include, but are not limited to, fluorescein/tetramethylrhodamine, IAEDANS/FITC, IAEDANS/5-(iodoacetomido)fluorescein, EDANS/Dabcyl, and B-phyco-erythrin/Cy-5.

The use of physically linked fluorescent reporter/quencher molecule pairs is also within the scope of the invention. The use of such systems in TaqMan™ assays (as described, for example, in U.S. Pat. Nos. 5,210,015; 5,804,375; 5,487,792 and 6,214,979) or as Molecular Beacons (as described, for example in, S. Tyagi and F. R. Kramer, Nature Biotechnol. 1996, 14: 303-308; S. Tyagi et al., Nature Biotechnol. 1998, 16: 49-53; L. G. Kostrikis et al., Science, 1998, 279: 1228-1229; D. L. Sokol et al., Proc. Natl. Acad. Sci. USA, 1998, 95: 11538-11543; S. A. Marras et al., Genet. Anal. 1999, 14: 151-156; and U.S. Pat. Nos. 5,846,726, 5,925,517, 6,277,581 and 6,235,504) is well-known in the art. With the TaqMan™ assay format, products of the amplification reaction can be detected as they are formed or in a so-called “real-time” manner. As a result, amplification product/probe hybrids are formed and detected while the reaction mixture is under amplification conditions.

In certain preferred embodiments of the present invention, the PCR detection probes are TaqMan™-like probes, i.e., probes having oligonucleotide sequences that are labeled at the 5′-end with a fluorescent moiety and at the 3′-end with a quencher moiety. Suitable fluorophores and quenchers for use with TaqMan™-like probes are disclosed, for example, in U.S. Pat. Nos. 5,210,015, 5,804,375, 5,487,792 and 6,214,979 and international application No. WO 01/86001. Examples of quenchers include, but are not limited to DABCYL (i.e., 4-(4′-dimethyl-aminophenylazo)-benzoic acid) succinimidyl ester, diarylrhodamine carboxylic acid, succinimidyl ester (or QSY-7), and 4′,5′-dinitrofluorescein carboxylic acid, succinimidyl ester (or QSY-33) (all available, for example, from Molecular Probes), quenched (Q1; available from Epoch Biosciences, Bothell, Wash.), or “Black hole quenchers” BHQ-1, BHQ-2, and BHQ-3 (available from BioSearch Technologies, Inc., Novato, Calif.). In certain preferred embodiments, the PCR detection probes are TaqMan™-like probes that are labeled at the 5′ end with FAM and at the 3′ end with a Black Hole Quencher.

A “tail” of normal or modified nucleotides (e.g., a universal tag sequence) can also be added to oligonucleotide probes for detectability purposes. A second hybridization with nucleic acid complementary to the tail and containing one or more detectable labels (such as, for example, fluorophores, enzymes or bases that have been radioactively labeled) or attached to a solid support (e.g., microparticles or arrays) allows visualization of the amplicon/probe hybrids (see, for example, the system commercially available from Enzo Biochem. Inc., New York, N.Y.). Another example of an assay with which the inventive oligonucleotides are useful is a signal amplification method such as that described in U.S. Pat. No. 5,124,246 (which is incorporated herein by reference in its entirety). In that method, the signal is amplified through the use of amplification multimers, polynucleotides which are constructed so as to contain a first segment that hybridizes specifically to the “tail” added to the oligonucleotide probes, and a multiplicity of identical second segments that hybridize specifically to a labeled probe. The degree of amplification is theoretically proportional to the number of iterations of the second segment. The multimers may be either linear or branched. Branched multimers may be in the shape of a fork or a comb.

The selection of a particular nucleic acid labeling technique will depend on the particular situation and will be governed by several factors, such as the ease and cost of the labeling method, the quality of sample labeling desired, the effects of the detectable moiety on the hybridization reaction (e.g., on the rate and/or efficiency of the hybridization process), the nature of the amplification method used, the nature of the detection system, the nature and intensity of the signal generated by the detectable label, and the like.

### Amplification of HBV Target Sequences Using Inventive Primers

The use of oligonucleotide sequences of the present invention to amplify HBV target sequences in test samples is not limited to any particular nucleic acid amplification technique or any particular modification thereof. In fact, the inventive oligonucleotide sequences can be employed in any of a variety of nucleic acid amplification methods well-known in the art (see, for example, A. R. Kimmel and S. L. Berger, Methods Enzymol. 1987, 152: 307-316; J. Sambrook et al., “Molecular Cloning: A Laboratory Manual”, 1989, 2^{nd} Ed., Cold Spring Harbour Laboratory Press: New York, N.Y.; “Short Protocols in Molecular Biology”, F. M. Ausubel (Ed.), 2002, 5^{th} Ed., John Wiley & Sons: Secaucus, N.J.).

Such well-known nucleic acid amplification methods include, but are not limited to the Polymerase Chain Reaction (or PCR, described in, for example, “PCR Protocols: A Guide to Methods and Applications”, M. A. Innis (Ed.), 1990, Academic Press: New York; “PCR Strategies”, M. A. Innis (Ed.), 1995, Academic Press: New York; “Polymerase chain reaction: basic principles and automation in PCR: A Practical Approach”, McPherson et al. (Eds.), 1991, IRL Press: Oxford; Saiki et al., Nature, 1986, 324: 163; and U.S. Pat. Nos. 4,683,195, 4,683,202 and 4,889,818, each of which is incorporated herein by reference in its entirety); and variations thereof including TaqMan™-based assays (Holland et al., Proc. Natl. Acad. Sci., 1991, 88: 7276-7280), and reverse transcriptase polymerase chain reaction (or RT-PCR, described in, for example, U.S. Pat. Nos. 5,322,770 and 5,310,652).

In PCR, a pair of primers is employed in excess to hybridize to the complementary strands of the target nucleic acid. The primers are each extended by a DNA polymerase using the target sequence as a template. The extension products become target themselves after dissociation (denaturation) from the original target strand. New primers are then hybridized and extended by the polymerase, and the cycle is repeated to exponentially increase the number of copies of target sequence molecules. Examples of DNA polymerases capable of producing primer extension primers in PCR reactions include, but are not limited to: E. coli DNA polymerase I, Klenow fragment of DNA polymerase I, T4 DNA polymerase, thermostable DNA polymerases isolated from Thermus aquaticus (Taq), available from a variety of sources (for example, Perkin Elmer), Thermus thermophilus (United States Biochemicals), Bacillus stereothermophilus (Bio-Rad), or Thermococcus litoralis (“Vent” polymerase, New England Biolabs). RNA target sequences may be amplified by reverse transcribing the mRNA into cDNA, and then performing PCR (RT-PCR), as described above. Alternatively, a single enzyme may be used for both steps as described in U.S. Pat. No. 5,322,770.

In addition to the enzymatic thermal amplification described above, well-known isothermal enzymatic amplification reactions can be employed to amplify HBV target sequences using oligonucleotide primers of the present invention (S. C. Andras et al., Mol. Biotechnol., 2001, 19: 29-44). These methods include, but are not limited to, Transcription-Mediated Amplification (or TMA, described in, for example, D. Y. Kwoh et al., Proc. Natl. Acad. Sci. USA, 1989, 86: 1173-1177; C. Giachetti et al., J. Clin. Microbiol., 2002, 40: 2408-2419; and U.S. Pat. No. 5,399,491); Self-Sustained Sequence Replication (or 3SR, described in, for example, J. C. Guatelli et al., Proc. Natl. Acad. Sci. USA, 1990, 87: 1874-1848; and E. Fahy et al., PCR Methods and Applications, 1991, 1: 25-33); Nucleic Acid Sequence Based Amplification (or NASBA, described in, for example, T. Kievits et al., J. Virol., Methods, 1991, 35: 273-286; and U.S. Pat. No. 5,130,238) and Strand Displacement Amplification (or SDA, described in, for example, G. T. Walker et al., PNAS, 1992, 89: 392-396; EP 0 500 224 A2). Each of the references cited in this paragraph is incorporated herein by reference in its entirety.

Strand-displacement amplification (SDA) combines the ability of a restriction endonuclease to nick the unmodified strand of its target DNA and the action of an exonuclease-deficient DNA polymerase to extend the 3′ end at the nick and displace the downstream DNA strand at a fixed temperature (G. T. Walker et al., Proc. Natl. Acad. Sci. USA, 1992, 89: 392-396). Primers used in SDA include a restriction endonuclease recognition at site 5′ to the target binding sequence (U.S. Pat. Nos. 5,270,184 and 5,344,166, each of which is incorporated herein by reference in its entirety). Nucleic Acid Sequence Based Amplification (NASBA) uses three enzymes—e.g., RNase H, avian myeloblastosis virus (AMV) reverse transcriptase and T7 RNA polymerase—working in concert at a low isothermal temperature, generally 41° C. (J. Compton, Nature, 1991, 350: 91-92; A. B. Chan and J. D. Fox, Rev. Med. Microbiol., 1999, 10: 185-196). The product of a NASBA reaction is mainly single-stranded RNA. The Self Sustaining Sequence Replication (3SR) reaction is a very efficient method for isothermal amplification of target DNA or RNA sequences. A 3SR system involves the collective activities of AMV reverse transcriptase, E. Coli RNase H, and DNA-dependent RNA polymerase (e.g., T7 RNA polymerase). Transcription-Mediated Amplification (TMA) uses an RNA polymerase to make RNA from a promoter engineered in the primer region, a reverse transcriptase to produce complementary DNA from the RNA templates and RNase H to remove the RNA from cDNA (J. C. Guatelli et al., Proc. Natl. Acad. Sci. USA, 1990, 87: 1874-1878).

NASBA, 3SR, and TMA primers require an RNA polymerase promoter linked to the target binding sequence of the primer. Promoters or promoter sequences for incorporation in the primers are nucleic acid sequences (either naturally occurring, produced synthetically or products of a restriction digest) that are specifically recognized by an RNA polymerase that binds to that sequence and initiates the process of transcription whereby RNA transcripts are generated. Examples of useful promoters include those which are recognized by certain bacteriophage polymerases such as those from bacteriophage T3, T7 or SP6 or a promoter from E. coli.

### Detection of Amplified HBV Target Sequences

In certain preferred embodiments of the present invention, oligonucleotide probe sequences are used to detect amplification products generated by the amplification reaction (i.e., amplified HBV target sequence(s)). The inventive probe sequences can be employed using a variety of well-known homogeneous or heterogeneous methodologies.

Homogeneous detection methods include, but are not limited to, the use of FRET labels attached to the probes that emit a signal in the presence of the target sequence, Molecular Beacons (S. Tyagi and F. R. Kramer, Nature Biotechnol. 1996, 14: 303-308; S. Tyagi et al., Nature Biotechnol. 1998, 16: 49-53; L. G. Kostrikis et al., Science, 1998, 279: 1228-1229; D. L. Sokol et al., Proc. Natl. Acad. Sci. USA, 1998, 95: 11538-11543; S. A. Marras et al., Genet. Anal. 1999, 14: 151-156; and U.S. Pat. Nos. 5,846,726, 5,925,517, 6,277,581 and 6,235,504), and so-called TaqMan™ assays (U.S. Pat. Nos. 5,210,015; 5,804,375; 5,487,792 and 6,214,979 and WO 01/86001). Using these detection techniques, products of the amplification reaction can be detected as they are formed or in a so-called real time manner. As a result, amplification product/probe hybrids are formed and detected while the reaction mixture is under amplification conditions.

In certain preferred embodiments, the detection probes of the present invention are used in a TaqMan™ assay. A TaqMan™ assay, also known as fluorogenic 5′ nuclease assay, is a powerful and versatile PCR-based detection system for nucleic acid targets. Analysis is performed in conjunction with thermal cycling by monitoring the generation of fluorescence signals. The assay system has the capability of generating quantitative data allowing the determination of target copy numbers. For example, standard curves can be produced using serial dilutions of previously quantified suspensions of HBV, against which unknown samples can be compared. The TaqMan™ assay is conveniently performed using, for example, AmpliTaq Gold™ DNA polymerase, which has endogenous 5′ nuclease activity, to digest an oligonucleotide probe labeled with both a fluorescent reporter dye and a quencher moiety, as described above. Assay results are obtained by measuring changes in fluorescence that occur during the amplification cycle as the probe is digested, uncoupling the fluorescent and quencher moieties and causing an increase in the fluorescence signal that is proportional to the amplification of the target sequence.

Other examples of homogeneous detection methods include hybridization protection assays (HPA). In such assays, the probes are labeled with acridinium ester (AE), a highly chemiluminescent molecule (Weeks et al., Clin. Chem., 1983, 29: 1474-1479; Berry et al., Clin. Chem., 1988, 34: 2087-2090), using a non-nucleotide-based linker arm chemistry (U.S. Pat. Nos. 5,585,481 and 5,185,439). Chemiluminescence is triggered by AE hydrolysis with alkaline hydrogen peroxide, which yields an excited N-methyl acridone that subsequently deactivates with emission of a photon. In the absence of a target sequence, AE hydrolysis is rapid. However, the rate of AE hydrolysis is greatly reduced when the probe is bound to the target sequence. Thus, hybridized and un-hybridized AE-labeled probes can be detected directly in solution, without the need for physical separation.

Heterogeneous detection systems are well-known in the art and generally employ a capture agent to separate amplified sequences from other materials in the reaction mixture. Capture agents typically comprise a solid support material (e.g., microtiter wells, beads, chips, and the like) coated with one or more specific binding sequences. A binding sequence may be complementary to a tail sequence added to the oligonucleotide probes of the invention. Alternatively, a binding sequence may be complementary to a sequence of a capture oligonucleotide, itself comprising a sequence complementary to a tail sequence of an inventive oligonucleotide probe. After separation of the amplification product/probe hybrids bound to the capture agents from the remaining reaction mixture, the amplification product/probe hybrids can be detected using any detection methods described above.

### II—Methods of Detection of HBV in Test Samples

In another aspect, the present invention provides methods for detecting the presence of HBV in a test sample. The inventive methods may be used to test patients who may or may not exhibit symptoms of HBV infection or its sequelae, and/or to screen at-risk populations.

Typically, certain methods of the invention comprise steps of: providing a test sample suspected of comprising HBV nucleic acids; contacting the test sample with at least one oligonucleotide disclosed herein, such that the oligonucleotide hybridizes to the HBV nucleic acid, if present in the test sample; and detecting any oligonucleotide hybridized to the HBV nucleic acid, wherein detection of an oligonucleotide hybridized to the HBV nucleic acid indicates the presence of HBV in the test sample.

Other methods of the invention comprise steps of: providing a test sample suspected of comprising a HBV nucleic acid; contacting the test sample with at least one primer set disclosed herein; amplifying all or a portion of the HBV nucleic acid using the primer set to obtain HBV amplicons; and detecting any HBV amplicons, wherein detection of HBV amplicons is indicative of the presence of HBV in the test sample.

Still other methods of the invention comprise steps of: providing a test sample suspected of comprising a HBV nucleic acid; contacting the test sample with at least one primer/probe set disclosed herein; amplifying all or a portion of the HBV nucleic acid using the primers of the primer/probe set to obtain HBV amplicons; detecting any HBV amplicons using the detection probe of the primer/probe set, wherein detection of HBV amplicons is indicative of the presence of HBV in the test sample.

Certain methods of the present invention may further comprise a step of quantifying any HBV amplicons to obtain the test sample's viral load. Oligonucleotide sequences and methods provided herein are such that they allow for a wide range of viral loads. In certain embodiments, oligonucleotide sequences and methods provided herein are such that they allow from less than about 50 to more than about 10⁸ copies/mL of HBV, to be quantified.

### Test Sample Preparation

According to methods provided by the present invention, the presence of HBV in a test sample can be determined by detecting a HBV nucleic acid comprising a sequence within the surface antigen gene of HBV. Thus, any liquid or solid biological material suspected of comprising such HBV target sequences can be a suitable test sample. Suitable test samples can include or be derived from blood, plasma, serum, urine, seminal fluid, saliva, lymphatic fluid, amniotic fluid, synovial fluid, peritoneal fluid, endocervical, urethral, rectal, vaginal, vulva-vaginal samples, and liver biopsy. In certain preferred embodiments, test samples comprise serum or plasma.

Test samples will often be obtained or isolated from patients suspected of being infected with HBV. A test sample may be used without further treatment after isolation or, alternatively, it may be processed before analysis. For example, a test sample may be treated so as to release HBV nucleic acids from the test sample. Methods of nucleic acid extraction are well-known in the art and include chemical methods, temperature methods, and mechanical methods (see, for example, J. Sambrook et al., “Molecular Cloning: A Laboratory Manual”, 1989, 2^{nd} Ed., Cold Spring Harbour Laboratory Press: New York, N.Y.). There are also numerous different and versatile kits that can be used to extract nucleic acids from biological samples that are commercially available from, for example, Amersham Biosciences (Piscataway, N.J.), BD Biosciences Clontech (Palo Alto, Calif.), Epicentre Technologies (Madison, Wis.), Gentra Systems, Inc. (Minneapolis, Minn.), MicroProbe Corp. (Bothell, Wash.), Organon Teknika (Durham, N.C.), and Qiagen Inc. (Valencia, Calif.). User Guides that describe in great detail the protocol to be followed are usually included in all these kits. Sensitivity, processing time and cost may be different from one kit to another. One of ordinary skill in the art can easily select the kit(s) most appropriate for a particular situation.

Prior to extraction, virions (infectious and noninfectious) and cells (including infected cells) may be purified, concentrated or otherwise separated from other components of the original biological sample, for example, by filtration or centrifugation.

### Sample Analysis

As will be appreciated by one skilled in the art, amplification of HBV target sequences and detection of amplified HBV nucleic acids according to methods of the present invention may be performed using any amplification/detection methodologies. In certain embodiments, detection of HBV in a test sample is performed using a TaqMan™ assay, and the formation of amplification products is monitored in a real time manner by fluorescence. In these embodiments, probes (e.g., comprising a HBV-specific sequence provided herein) will be used that are labeled with a fluorescent reporter at the 5′ end and a quencher moiety at the 3′ end, as described above. Optimization of amplification conditions and selection of amplification reaction reagents suitable for a TaqMan™ assay format are within the skill in the art.

In certain embodiments, an internal control or an internal standard is added to the biological sample (or to purified nucleic acids extracted from the biological sample) to serve as a control for extraction and/or target amplification. Preferably, the internal control includes a sequence that differs from the target sequence(s), and is capable of amplification by the primers used to amplify the target HBV nucleic acids. Alternatively, the internal control may be amplified by primers that are different from the primers used to amplify the target HBV nucleic acids. The use of an internal control allows monitoring of the isolation/extraction process, amplification reaction, and detection, and control of the assay performance. The amplified control and amplified target are typically distinguished at the detection step by using different probes (e.g., labeled with different detectable agents) for the detection of the control and target.

The presence of HBV in a test sample may be confirmed by repeating an assay according to the present invention using a different aliquot of the same biological test sample or using a different test sample (e.g., an endocervical swab if the first sample analyzed was a serum sample, or a serum sample collected at a different time). Alternatively or additionally, the presence of HBV in a test sample may be confirmed by performing a different assay (i.e., an assay using the same primer/probe set(s) but a different nucleic acid amplification methodology). For example, if the first analysis was performed using a TaqMan™ assay, a second analysis may be carried out using a transcription-mediated amplification (TMA) reaction or a conventional PCR reaction.

The presence of HBV in a test sample may, alternatively, be confirmed by a different assay, for example using a commercial HBV NAT detection kit or an immunological method.

### Quantitation

Certain methods of the present invention include determining the amount of HBV nucleic acid present in the sample obtained from the individual being tested. As will be appreciated by one skilled in the art, such determination can be performed using any suitable method. In certain embodiments, quantitation of HBV nucleic acid in a sample according to the present invention is performed using Real-Time PCR. Real-Time PCR methods include, but are not limited to, TaqMan®, Molecular Beacons®, Scorpions®, and SYBR® Green methods. All of these methods allow detection and quantitation of PCR products via the generation of a fluorescent signal. In certain embodiments, a TaqMan method is used.

As is well-known in the art, results obtained by Real Time PCR can be quantified using, for example, the standard curve method. In a standard curve method, a standard curve is first constructed from samples of HBV of known concentrations. This curve is then used as a reference standard for extrapolating quantitative information for samples of unknown concentrations.

### III—Uses of Inventive Oligonucleotide Sequences and Detection Methods

The invention provides a variety of assays for detecting/quantifying HBV present in a biological sample obtained from an individual. Such assays can be used in different applications including, but not limited to, the screening of at-risk groups or individuals as well as in the management of HBV infection in chronic HBV carriers.

### Screening of at Risk Groups and Individuals

HBV is generally transmitted horizontally by blood and blood products, and by sexual contact. It is also transmitted vertically from mother to infant in the perinatal period—this is a major mode of transmission in regions where hepatitis B is endemic. The blood supply in many countries has been screened for HBV for many years and, as a result, transmission by blood transfusion is extremely rare. Health care workers and patients receiving hemodialysis are also at increased risk of infection. Major routes of transmission among adults in Western countries are intravenous drug use and sexual contact. The most common risk factors for sexual transmission among heterosexuals include having multiple sexual partners, a history of sexually transmitted disease, or sex with a known infected person. Men who have sex with men are also at high risk of HBV transmission.

Assays of the present invention may be used for screening/testing at risk groups and at risk individuals including newborns of HBV carrier mothers, children under 10 years in high prevalence communities (e.g., children from countries where hepatitis B is endemic), household and sexual contacts of acute and chronic hepatitis B carriers, people at risk of sexual transmission, injecting drug users, hemodialysis patients, people with chronic liver disease, health care workers, recipients of certain blood products, international travelers who may have had sexual or blood exposures, and patients with damaged immune systems.

Assays of the present invention may also be used for testing individuals that exhibit symptoms of HBV infection, such as jaundice, fatigue, abdominal pain, loss of appetite, nausea, vomiting, and join pain; and individuals with abnormal laboratory tests suggesting liver disease.

### HBV Diagnosis

Practicing certain methods of the present invention includes providing a HBV diagnosis for the individual from whom the biological sample analyzed has been obtained. In certain embodiments, the HBV diagnosis is provided based on the detection of HBV nucleic acids in the biological sample tested. In other embodiments, the HBV diagnosis is provided based on the quantification of HBV nucleic acids in the sample. Providing a HBV diagnosis according to the present invention may include one or more of: determining if an individual is infected with HBV, determining a HBV infection stage for the individual, determining if the individual is afflicted with a HBV disease, determining the severity of a HBV disease afflicting the individual, determining the progression of a HBV disease afflicting the individual, determining the likelihood that an individual has to develop a HBV disease, and determining the efficacy of a HBV therapy in an individual.

In order to provide a diagnosis, HBV nucleic acid amounts or viral loads determined according to the present invention in biological samples obtained from individuals to be tested can be compared to amounts or viral loads determined in reference samples. Reference samples may be obtained from healthy individuals and/or from HBV-infected individuals diagnosed with a specific stage of HBV infection (e.g., highly replicative HBV infection stage, chronic HBV infection stage, late phase of chronic HBV infection stage, occult stage of HBV infection), a specific HBV disease (e.g., acute hepatitis, chronic hepatitis, cirrhosis, liver failure, hepatocellular carcinoma) or with a specific stage/advancement of the HBV disease. Reference HBV nucleic acid amounts or viral loads are preferably averages or means of amounts or viral loads determined for a significant number of individuals afflicted with the same HBV condition (e.g., same HBV disease with same degree of advancement of the disease).

Reference samples may also be obtained from individuals whose HBV infection/disease diagnosis, antiviral therapy history (e.g., drug response) and clinical outcome are known. Such samples may be used to determine reference HBV nucleic acid amounts or viral loads indicative of a risk of developing a given HBV disease or of reacting favorably or unfavorably to a specific therapeutic regimen.

It will be appreciated by one skilled in the art that the results obtained using methods according to the present invention may be compared to and/or combined with results from other tests, assays or procedures performed for the diagnosis of HBV infection and/or HBV disease. Such comparison and/or combination may help to provide a more thorough diagnosis and to guide individualized therapy.

Thus, for example, individuals tested using methods of the present invention may also undergo HBsAg testing and/or HBeAg testing. Liver damage may be diagnosed according to established techniques. For example, liver damage is often diagnosed using a set of clinical biochemistry laboratory blood assays designed to provide information about the state of the individual's liver. Since the liver produces most of the plasma proteins in the body, measuring the amount of total protein and/or the amount of albumin (the main constituent of total protein and a protein made specifically by the liver) in the blood gives information regarding the functioning state of the liver. When the liver is damaged, it may fail to produce blood clotting factors: the prothrombin time may be measured to diagnose disorders of blood clotting, usually bleeding, resulting from liver damage. Serum bilirubin concentration may also be measured as an indication of the individual's liver state. Bilirubin is the major breakdown product that results from the destruction of old red blood cells (as well as other sources). It is removed from the blood by the liver, chemically modified by a process called conjugation, secreted into the bile, passed into the intestine and to some extent reabsorbed from the intestine. Many different liver diseases and conditions can cause the serum bilirubin concentrations to be elevated. Blood assays may also be performed to measure one or more of alanine transaminase (ALT), alkaline phosphatase (ALP), aspartate transaminase (AST), and gamma glutamyl transpeptidase (GGT). ALT is an enzyme present in hepatocytes. When a hepatic cell is damaged, it leaks this enzyme into the blood, where it can be measured. ALT rises dramatically in acute liver damage, such as viral hepatitis. Elevations are often measured in multiples of the upper limit of normal (ULN). ALP is an enzyme present in cells lining the biliary ducts of the liver. AST is similar to ALT in that it is another enzyme associated with parenchymal cells, that is raised in acute liver damage. GGT is an enzyme whose levels may be elevated with even minor, sub-clinical levels of liver dysfunction. An individual tested using methods of the present invention may also undergo liver biopsy, ultrasound of the liver, and/or alpha-fetoprotein (AFP) testing.

### Selection of Appropriate Treatment and Treatment Monitoring

Using assays disclosed herein, skilled physicians may select and prescribe treatment adapted to each individual based on the diagnosis and HBV infection staging provided to the individual through determination of the HBV viral load. Methods of the present invention can also be used to monitor the course of HBV disease therapy. Thus, for example, by measuring the increase or decrease of HBV nucleic acids in a biological sample (e.g., viral load in serum or plasma) according to the present invention, it is possible to determine whether a particular therapeutic regimen aimed at ameliorating HBV disease is effective. One of the advantages of certain inventive methods is that they provide physicians with a means to quantify very low (i.e., less than about 50 HBV copies/mL) to very high (i.e., more than about 10⁸ HBV copies/mL) HBV viral loads using a single test.

Selection of an appropriate therapeutic regimen for a given patient may be made solely on the diagnosis/staging provided by one of the inventive methods. Alternatively, the physician may also consider other clinical or pathological parameters used in existing methods to diagnose HBV infection and/or HBV disease and assess its advancement, as described above.

Interferons-alpha were the first drugs approved in the U.S. for the treatment of chronic hepatitis B. Benefits to interferon therapy include a short, defined treatment duration of 4 months to 1 year, lack of viral resistance, and low relapse rates. Interferon therapy, however, is costly, requires subcutaneous injection, has clinically significant side effects (including depression) and results in durable virologic response in only 15% to 30% of patients (D. K. Wong et al., Ann. Intern. Med., 1993, 119: 312-323; C. Niederau et al., New Engl. J. Med., 1996, 334: 1422-1427; G. V. Papatheodoridis et al., J. Hepatol., 2001, 34: 306-313; G. Fattavich et al., Hepatotol., 1997, 26: 1338-1342). Other treatment options for chronic hepatitis B include nucleoside analogues, such as lamivudine, approved in December 1998 by the U.S. Food and Drug Administration (FDA), adevofir dipivoxil approved in September 2002 by the FDA, and entecavir (Baraclude) approved Mar. 30, 2005 by the FDA. Entecavir, at least, appears to have the ability to drop HBV viral load by four logs. In addition to being approved for the treatment of chronic hepatitis B, both lamivudine and adevofir dipivoxil are effective against HIV. Nucleoside analogues are easy to administer and are associated with less side effects than interferon-α (Y. F. Liaw et al., Gastroenterol., 2000, 119: 172-180; B. C. Song et al., Hepatol., 2000, 32: 803-805; J. L. Dienstag et al., Hepatol., 1999, 30: 1082-1087). However, they have a high rate of viral resistance (M. Atkins et al., Hepatol., 1998, 28: 319A) and exhibit lower durable response rates and a greater need for prolonged therapy compared to interferon (Y. F. Liaw et al., Gastroenterol., 2000, 119: 172-180; B. C. Song et al., Hepatol., 2000, 32: 803-805; J. L. Dienstag et al., Hepatol., 1999, 30: 1082-1087).

Thus, accurate diagnosis of HBV infection and HBV infection staging is important to make the decision to initiate antiviral treatment and select an appropriate therapeutic regimen. Furthermore, close monitoring of patients during and after antiviral therapy is also important due to the development of genotypic resistance, which causes virologic breakthrough (i.e., rise in serum HBV DNA levels) and occurrence of hepatitis flares withdrawal from antiviral therapy. Assays of the present invention can be used for both HBV diagnosis and HBV treatment monitoring and prognosis.

### IV—Kits

In another aspect, the present invention provides kits comprising materials useful for the detection of HBV according to methods described herein. The inventive kits may be used by diagnostic laboratories, experimental laboratories, or practitioners.

Basic materials and reagents for the detection of HBV according to the present invention may be assembled together in a kit. In certain embodiments, the kit comprises at least one inventive primer set or primer/probe set, and optionally amplification reaction reagents. Each kit preferably comprises the reagents which render the procedure specific. Thus, a kit adapted for use with NASBA preferably contains primers with RNA polymerase promoter linked to the target binding sequence, while a kit adapted for use with SDA preferably contains primers including a restriction endonuclease recognition site 5′ to the target binding sequence. Similarly, when a kit is adapted for use in a 5′ nuclease assay, such as the TaqMan™ assay, the detection probe(s) preferably contain(s) at least one fluorescent reporter moiety and at least one quencher moiety.

Suitable amplification reaction reagents include, for example, one or more of: buffers, enzymes having reverse transcriptase and/or polymerase activity or exonuclease activity; enzyme cofactors such as magnesium or manganese; salts; nicotinamide adenide dinuclease (NAD); and deoxynucleoside triphosphates (dNTPs) such as, for example, deoxyadenosine triphospate; deoxyguanosine triphosphate, deoxycytidine triphosphate and thymidine triphosphate suitable for carrying out the amplification reaction. For example, a kit, adapted for use with NASBA, may contain suitable amounts of reverse transcriptase, RNase H and T7 RNA polymerase. In kits adapted for transcription amplification reactions, such as NASBA, buffers can be included that contain, for example, DMSO, which is known to enhance the amplification reaction.

Depending on the procedure, the kit may further comprise one or more of: wash buffers and/or reagents, hybridization buffers and/or reagents, labeling buffers and/or reagents, and detection means. The buffers and/or reagents are preferably optimized for the particular amplification/detection technique for which the kit is intended. Protocols for using these buffers and reagents for performing different steps of the procedure may also be included in the kit.

Furthermore, kits may be provided with an internal control as a check on the sample preparation and amplification procedures and to prevent occurrence of false negative test results due to failures in the extraction or amplification procedures. An optimal control sequence is selected in such a way that it will not compete with the target nucleic acid sequence in the amplification reaction (as described above).

Kits may also contain reagents for the isolation of nucleic acids from biological specimen prior to amplification.

Reagents included in an inventive kit may be supplied in a solid (e.g., lyophilized) or liquid form. The kits may optionally comprise different containers (e.g., vial, ampoule, test tube, flask or bottle) for each individual buffer and/or reagent. Each component will generally be suitable as aliquoted in its respective container or provided in a concentrated form. Other containers suitable for conducting certain steps of the amplification/detection assay may also be provided. The individual containers of the kit are preferably maintained in close confinement for commercial sale.

The kit may also comprise instructions for using the amplification reaction reagents and primer sets or primer/probe sets according to the present invention. Instructions for using the kit according to one or more methods of the invention may comprise instructions for processing the biological sample, extracting nucleic acid molecules, and/or performing the test; instructions for interpreting the results, as well as a notice in the form prescribed by a governmental agency (e.g., FDA) regulating the manufacture, use or sale of pharmaceuticals or biological products.

### EXAMPLES

The following examples describe some of the preferred modes of making and practicing the present invention. However, it should be understood that these examples are for illustrative purposes only and are not meant to limit the scope of the invention. Furthermore, unless the description in an Example is presented in the past tense, the text, like the rest of the specification, is not intended to suggest that experiments were actually performed or data were actually obtained.

### General Information

The experiments described in the Examples presented below were performed using a set of primers comprising the forward amplification primer, Fp101 and reverse amplification primer, Rp276 and probe P225 (see the table presented in FIG. 1 for sequences). Testing of panels and samples was carried out using the Microlab® Starlet instrument (Hamilton Life Science Robotics, Reno, Nev.) for sample preparation and the Stratagene Mx3000P (Stratagene, La Jolla, Calif.) for amplification and detection.

### Example 1

### Genotype Equivalence

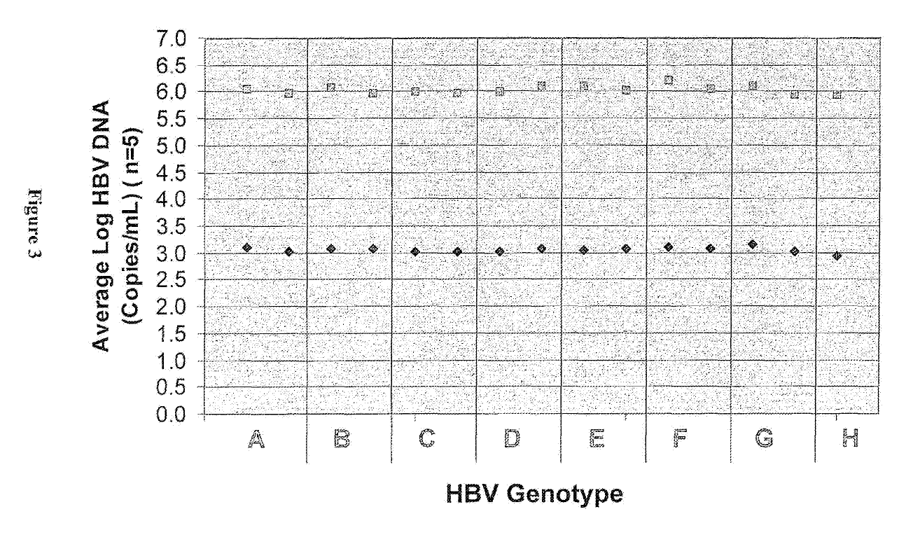

To demonstrate that the inventive oligonucleotide sequences recognize all eight genotypes of HBV, genotype equivalence was assessed using 15 plasmid DNA representing 8 HBV genotypes. Two isolates of each of the 7 HBV genotypes A-G and 1 isolate of genotype H were tested at a low level of 1000 DNA copies per PCR reaction and a high target concentration of 1,000,000 DNA copies per PCR reaction. Copy number for each HBV genotype plasmid sample was determined using the Versant HBV DNA 3.0 Assay (bDNA assay). Each genotype sample was tested using five replicates. These samples were tested across 2 runs with 96 specimens tested in each run. Results obtained are presented on FIG. 3. They show that genotypes B-H were quantitated on average within ±0.5 log relative to genotype A.

### Example 2

### Linearity and Detection Limits

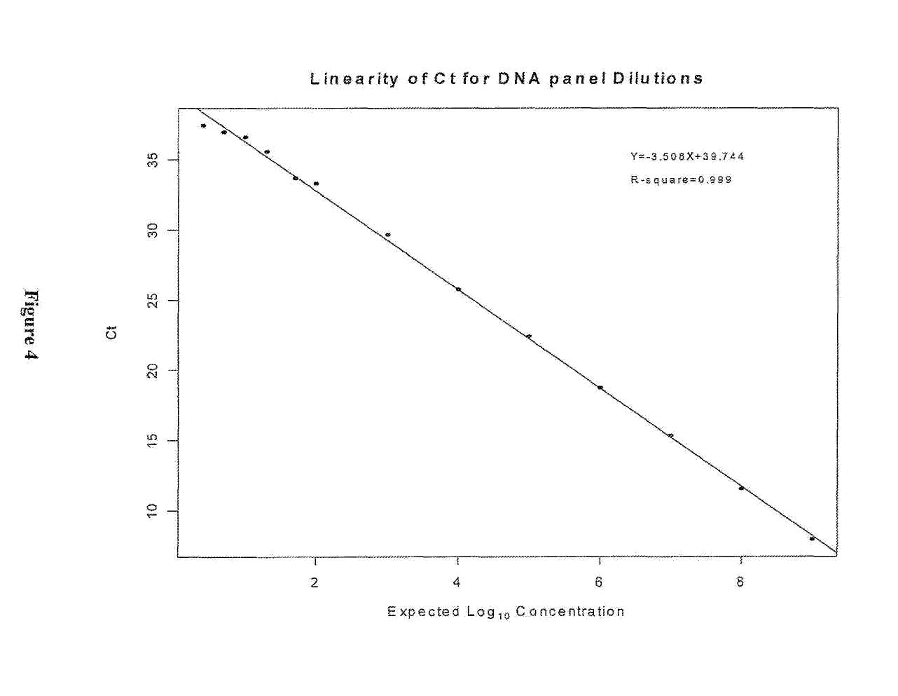

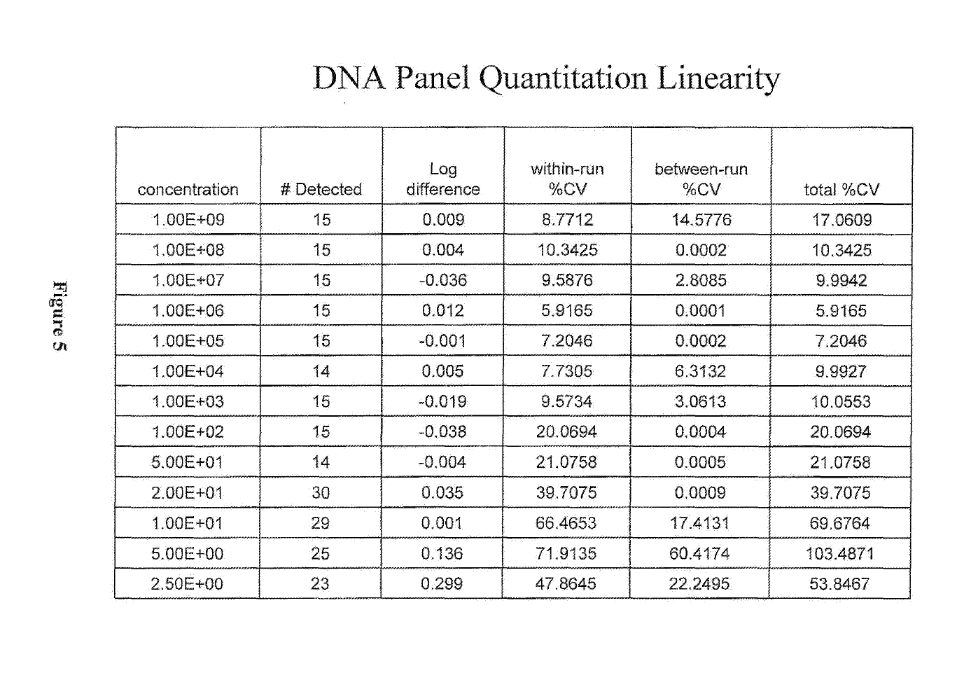

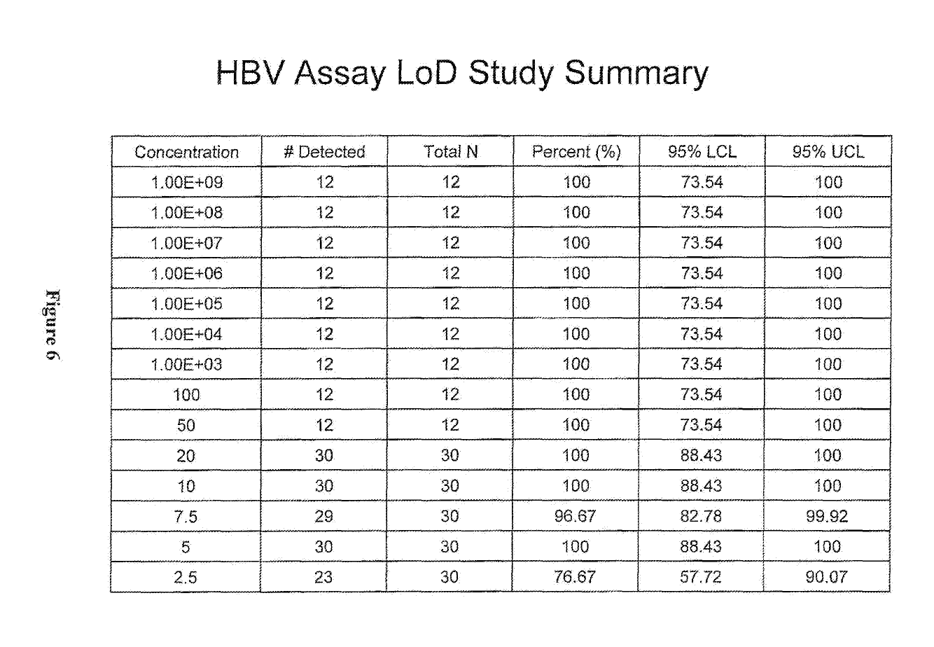

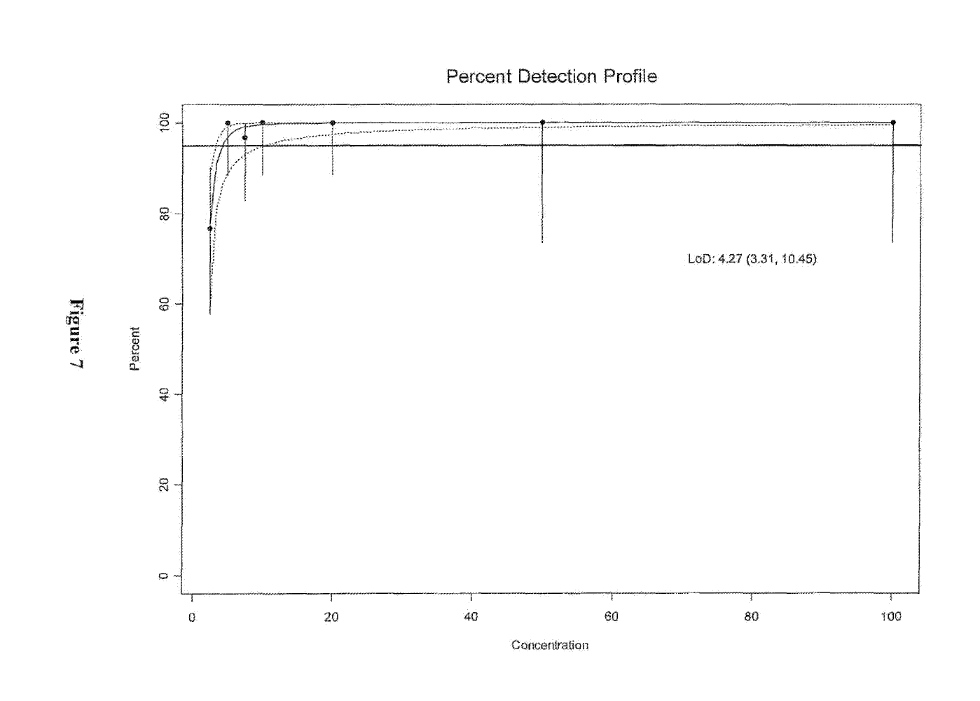

Determination of quantitation linear range and detection limits (LoD) of oligonucleotides of the present invention was performed using HBV recombination DNA fragments (rDNA) as target. HBV rDNA was quantitated by phosphate analysis. Results of linearity detection are presented on FIG. 4 and FIG. 5. Since one would expect |log diff| to be ≦0.1 and % CV to be as small as possible, the linear range can be determined to be from 10 copies per reaction to 1×10⁹ copies per reaction. Results of the determination of detection limits are presented on FIG. 6 and FIG. 7. LoD was determined to be 4.27 copies per reaction.

### Example 3

### Specificity

No cross-hybridization was observed with HIV, as was demonstrated by testing 10⁸ copies per PCR reaction of HIV transcript and 6 HIV positive clinical specimens. Similarly, no cross-hybridization was observed with HCV. Three hundred (300) unique HBsAg negative specimens, representing 150 serum and 150 EDTA plasma samples were tested to evaluate assay specificity. The final specificity was 100% (based on N=300) with lower one-sided 95% confidence limit equal to 99.01%.

### Example 4

### Linearity and Detection Limits on Clinical Specimens

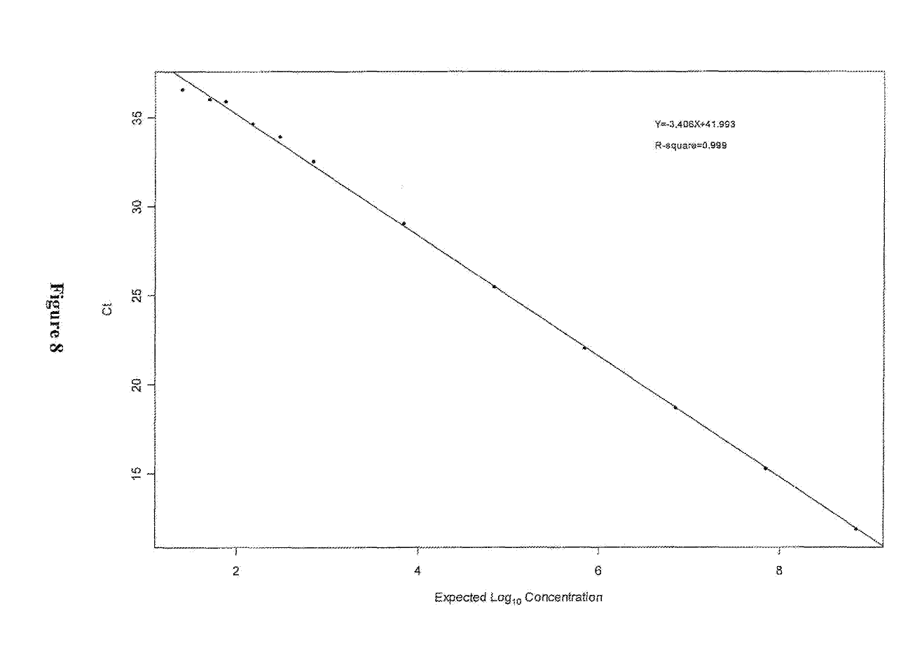

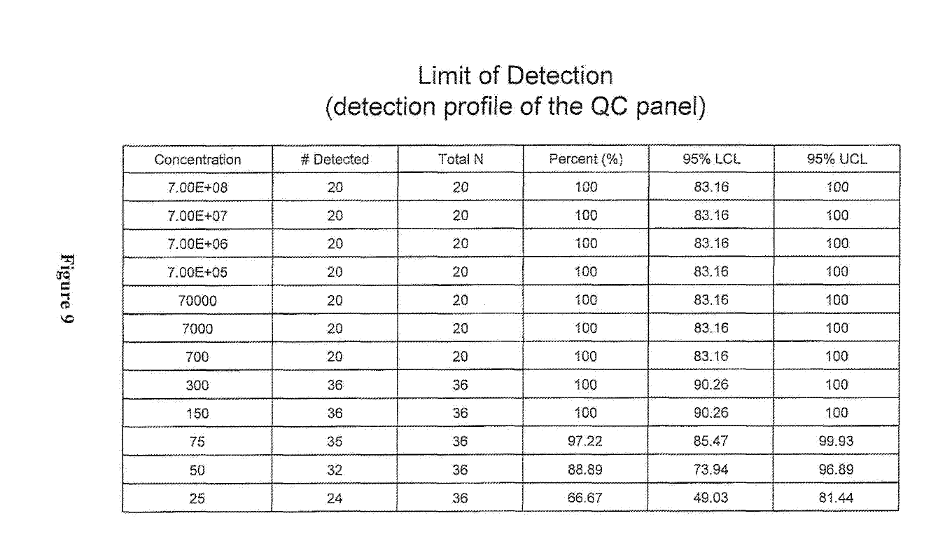

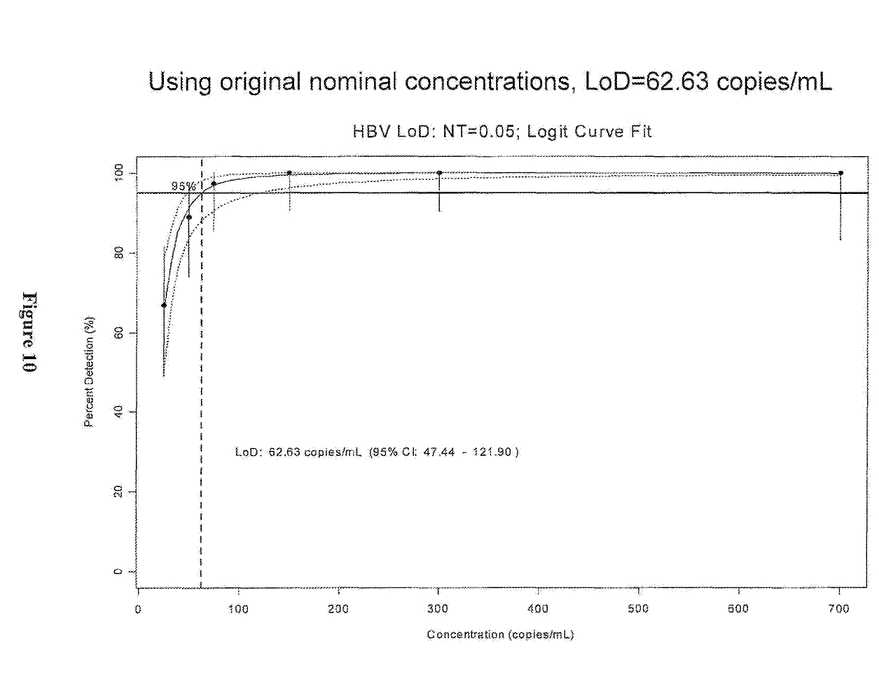

Determination of quantitation linear range and detection limits (LoD) of oligonucleotides of the present invention was performed using HBV clinical specimens. Clinical samples were extracted using the Microlab® Starlet instrument, and amplified using Stratagene Mx3000P. DNA copies were determined using the Versant HBV DNA 3.0 Assay (bDNA assay). Results of linearity detection are presented on FIG. 8. The linear range was determined to be from 7×10⁸ copies/mL to 50 copies/mL. Results of the determination of detection limits are presented on FIG. 9 and FIG. 10. LoD was determined to be less than 70 copies/mL (value assigned by bDNA assay).

### Other Embodiments

Other embodiments of the invention will be apparent to those skilled in the art from a consideration of the specification or practice of the invention disclosed herein. It is intended that the specification and examples be considered as exemplary only, with the true scope of the invention being indicated by the following claims.

1. An isolated oligonucleotide having a sequence selected from the group consisting of SEQ ID NOs. 1-9, complementary sequences thereof, active fragments thereof, and combinations thereof.

2. An isolated oligonucleotide amplification primer having a sequence selected from the group consisting of SEQ ID NOs. 1-5, complementary sequences thereof, active fragments thereof, and combinations thereof.

3. An isolated oligonucleotide detection probe having a sequence selected from the group consisting of SEQ ID NOs. 6-9, complementary sequences thereof, active fragments thereof, and combinations thereof.

4. An isolated oligonucleotide detection probe of claim 4 further comprising a detectable label.

5. The oligonucleotide detection probe of claim 4, wherein the detectable label comprises a fluorescent moiety attached at the 5′ end of the oligonucleotide.

6. The oligonucleotide detection probe of claim 5, wherein said oligonucleotide further comprises a quencher moiety attached to its 3′ end.

7. The oligonucleotide detection probe of claim 6, wherein the fluorescent moiety comprises 6-carboxyfluorescein and the quencher moiety comprises a Black Hole Quencher.

8. A collection of oligonucleotides for detecting HBV in a test sample, the collection comprising at least one primer set selected from the group consisting of: Primer Set 1, Primer Set 2, and Primer Set 3, wherein:
Primer Set 1 comprises a forward primer having a sequence as set forth in SEQ ID NO. 1 or any active fragment thereof, and a reverse primer having a sequence as set forth in SEQ ID NO. 4 or any active fragment thereof;
Primer Set 2 comprises a forward primer having a sequence as set forth in SEQ ID NO. 2 or any active fragment thereof, and a reverse primer having a sequence as set forth in SEQ ID NO. 4 or any active fragment thereof; and
Primer Set 3 comprises a forward primer having a sequence as set forth in SEQ ID NO. 3 or any active fragment thereof, and a reverse primer having a sequence as set forth in SEQ ID NO. 5 or any active fragment thereof.

9. A collection of oligonucleotides for detecting HBV in a test sample, the collection comprising at least one primer/probe set selected from the group consisting of: Primer/Probe Set 1(a), Primer/Probe Set 1(b), Primer/Probe Set 1(c), Primer/Probe Set 2(a), Primer/Probe Set 2(b), Primer/Probe Set 2(c), and Primer/Probe Set 3, wherein:
Primer/Probe 1(a) comprises a forward primer having a sequence as set forth in SEQ ID NO. 1 or any active fragment thereof, a reverse primer having a sequence as set forth in SEQ ID NO. 4 or any active fragment thereof, and a detection probe having a sequence as set forth in SEQ ID NO. 6 or any active fragment thereof;
Primer/Probe Set 1(b) comprises a forward primer having a sequence as set forth in SEQ ID NO. 1 or any active fragment thereof, a reverse primer having a sequence as set forth in SEQ ID NO. 4 or any active fragment thereof, and a detection probe having a sequence as set forth in SEQ ID NO. 7 or any active fragment thereof;
Primer/Probe Set 1(c) comprises a forward primer having a sequence as set forth in SEQ ID NO. 1 or any active fragment thereof, a reverse primer having a sequence as set forth in SEQ ID NO. 4 or any active fragment thereof, and a detection probe having a sequence as set forth in SEQ ID NO. 8 or any active fragment thereof;
Primer/Probe Set 2(a), comprises a forward primer having a sequence as set forth in SEQ ID NO. 2 or any active fragment thereof, a reverse primer having a sequence as set forth in SEQ ID NO. 4 or any active fragment thereof, and a detection probe having a sequence as set forth in SEQ ID NO. 6 or any active fragment thereof;
Primer/Probe Set 2(b) comprises a forward primer having a sequence as set forth in SEQ ID NO. 2 or any active fragment thereof, a reverse primer having a sequence as set forth in SEQ ID NO. 4 or any active fragment thereof, and a detection probe having a sequence as set forth in SEQ ID NO. 7 or any active fragment thereof;
Primer/Probe Set 2(c) comprises a forward primer having a sequence as set forth in SEQ ID NO. 2 or any active fragment thereof, a reverse primer having a sequence as set forth in SEQ ID NO. 4 or any active fragment thereof, and a detection probe having a sequence as set forth in SEQ ID NO. 8 or any active fragment thereof; and
Primer/Probe Set 3 comprises a forward primer having a sequence as set forth in SEQ ID NO. 3 or any active fragment thereof, a reverse primer having a sequence as set forth in SEQ ID NO. 5 or any active fragment thereof, and a detection probe having a sequence as set forth in SEQ ID NO. 9 or any active fragment thereof.

10. The collection of oligonucleotides of claim 9, wherein the detection probe of the at least one primer/probe set comprises a detectable label.

11. The collection of oligonucleotides of claim 10, wherein the detectable label comprises a fluorescent moiety attached at the 5′ end of the detection probe.

12. The collection of oligonucleotides of claim 11, wherein the detection probe further comprises a quencher moiety attached at its 3′ end.

13. The collection of oligonucleotides of claim 12, wherein the fluorescent moiety comprises 6-carboxyfluorescein and the quencher moiety comprises a Black Hole Quencher.

14. A kit for detecting HBV in a test sample, comprising:
amplification reaction reagents; and
at least one primer set selected from the group consisting of: Primer Set 1, Primer Set 2, and Primer Set 3, wherein:
Primer Set 1 comprises a forward primer having a sequence as set forth in SEQ ID NO. 1 or any active fragment thereof, and a reverse primer having a sequence as set forth in SEQ ID NO. 4 or any active fragment thereof;
Primer Set 2 comprises a forward primer having a sequence as set forth in SEQ ID NO. 2 or any active fragment thereof, and a reverse primer having a sequence as set forth in SEQ ID NO. 4 or any active fragment thereof; and
Primer Set 3 comprises a forward primer having a sequence as set forth in SEQ ID NO. 3 or any active fragment thereof, and a reverse primer having a sequence as set forth in SEQ ID NO. 5 or any active fragment thereof.

15. A kit for detecting HBV in a test sample, comprising:
amplification reaction reagents; and
at least one primer/probe Set selected from the group consisting of: Primer/Probe Set 1(a), Primer/Probe Set 1(b), Primer/Probe Set 1(c), Primer/Probe Set 2(a), Primer/Probe Set 2(b), Primer/Probe Set 2(a), and Primer/Probe Set 3, wherein:
Primer/Probe 1(a) comprises a forward primer having a sequence as set forth in SEQ ID NO. 1 or any active fragment thereof, a reverse primer having a sequence as set forth in SEQ ID NO. 4 or any active fragment thereof, and a detection probe having a sequence as set forth in SEQ ID NO. 6 or any active fragment thereof;
Primer/Probe Set 1(b) comprises a forward primer having a sequence as set forth in SEQ ID NO. 1 or any active fragment thereof, a reverse primer having a sequence as set forth in SEQ ID NO. 4 or any active fragment thereof, and a detection probe having a sequence as set forth in SEQ ID NO. 7 or any active fragment thereof;
Primer/Probe Set 1(c) comprises a forward primer having a sequence as set forth in SEQ ID NO. 1 or any active fragment thereof, a reverse primer having a sequence as set forth in SEQ ID NO. 4 or any active fragment thereof, and a detection probe having a sequence as set forth in SEQ ID NO. 8 or any active fragment thereof;
Primer/Probe Set 2(a), comprises a forward primer having a sequence as set forth in SEQ ID NO. 2 or any active fragment thereof, a reverse primer having a sequence as set forth in SEQ ID NO. 4 or any active fragment thereof, and a detection probe having a sequence as set forth in SEQ ID NO. 6 or any active fragment thereof;
Primer/Probe Set 2(b) comprises a forward primer having a sequence as set forth in SEQ ID NO. 2 or any active fragment thereof, a reverse primer having a sequence as set forth in SEQ ID NO. 4 or any active fragment thereof, and a detection probe having a sequence as set forth in SEQ ID NO. 7 or any active fragment thereof;
Primer/Probe Set 2(c) comprises a forward primer having a sequence as set forth in SEQ ID NO. 2 or any active fragment thereof, a reverse primer having a sequence as set forth in SEQ ID NO. 4 or any active fragment thereof, and a detection probe having a sequence as set forth in SEQ ID NO. 8 or any active fragment thereof; and
Primer/Probe Set 3 comprises a forward primer having a sequence as set forth in SEQ ID NO. 3 or any active fragment thereof, a reverse primer having a sequence as set forth in SEQ ID NO. 5 or any active fragment thereof, and a detection probe having a sequence as set forth in SEQ ID NO. 9 or any active fragment thereof.

16. The kit of claim 15, wherein the detection probe of the at least one primer/probe set at comprises a detectable label.

17. The kit of claim 16, wherein the detectable label comprises a fluorescent moiety attached at the 5′ end of the detection probe.

18. The kit of claim 17, wherein the detection probe further comprises a quencher moiety attached at its 3′ end.

19. The kit of claim 18, wherein the fluorescent moiety comprises 6-carboxyfluorescein and the quencher moiety comprises a Black Hole Quencher.

20. A method for detecting HBV in a test sample, the method comprising steps of:
providing a test sample suspected of comprising a HBV nucleic acid;
contacting the test sample with a least one oligonucleotide of claim 1 such that the at least one oligonucleotide hybridizes to the HBV nucleic acid, if present in the test sample; and
detecting any oligonucleotide hybridized to the HBV nucleic acid, where detection of an oligonucleotide hybridized to the HBV nucleic acid indicates the presence of HBV in the test sample.

21. The method of claim 20, wherein the step of detecting comprises steps of: amplifying all or a portion of the HBV nucleic acid to obtain HBV amplicons, and detecting any HBV amplicons.

22. The method of claim 21, wherein the step of amplifying is performed using polymerase chain reaction (PCR), Reverse-Transcriptase PCR (RT-PCR), or a Taq-Man™ assay.

23. A method for detecting HBV in a test sample, the method comprising steps of:
providing a test sample suspected of comprising HBV nucleic acid;
contacting the test sample with a least one primer set of the collection of oligonucleotides of claim 8;
amplifying all or a portion of the HBV nucleic acid using the primer set to obtain HBV amplicons; and
detecting any HBV amplicons, wherein detection of HBV amplicons is indicative of the presence of HBV in the test sample.

24. The method of claim 23, wherein the step of amplifying is performed using polymerase chain reaction (PCR), Reverse-Transcriptase PCR (RT-PCR), or a Taq-Man™ assay.

25. A method for detecting HBV in a test sample, the method comprising steps of:
providing a test sample suspected of comprising HBV nucleic acid;
contacting the test sample with a least one primer/probe set of the collection of oligonucleotides of claim 9;
amplifying all or a portion of the HBV nucleic acid using the primers of the primer/probe set to obtain HBV amplicons; and
detecting any HBV amplicons using the detection probe of the primer/probe set, wherein detection of HBV amplicons is indicative of the presence of HBV in the test sample.

26.-37. (canceled)
